SEQUENCE LISTING (33 sequences)

SEQ ID NO: 1 - protein, 860 aa, Aspergillus aculeatus
  BGL1 gene
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 2 - DNA, 2664 bp, Artificial Sequence
  Description of Artificial Sequence Synthetic modified AaBGL1 gene polynucleotide
cgaattggcggaaggccgtcaaggccgcatggtacctcatgaagttgtcctggcttgaggccgctgcccttaccgccgcctccgtcgtttccgccgacgagttggccttctccccccccttctacccctccccctgggccaacggtcaaggtgagtgggccgaggcttaccaacgtgccgtcgccattgtctcccaaatgacccttgacgagaaggtcaaccttaccaccggtactggttgggagcttgagaagtgcgtcggtcaaaccggtggtgtcccccgtcttaacatcggtggtatgtgccttcaagactccccccttggtatccgtgactccgactacaactccgccttccctgccggtgtcaacgtcgccgccacctgggacaagaaccttgcctaccttcgtggtcaagctatgggtcaagagttctccgacaagggtatcgacgtccaacttggtcctgccgccggtccccttggtcgttcccccgacggtggtcgtaactgggagggtttctcccccgaccccgcccttaccggtgtccttttcgccgagactatcaagggtatccaagacgctggtgtcgtcgccaccgccaagcactacatccttaacgagcaagagcacttccgtcaagtcgccgaggccgctggttacggtttcaacatctccgacaccatctcttccaacgttgacgacaagaccatccacgagatgtacctttggcccttcgccgacgccgtccgtgccggtgtcggtgccatcatgtgctcctacaaccaaatcaacaactcctacggttgccaaaactcctacacccttaacaagttgttgaaggccgagcttggtttccaaggtttcgtcatgtccgactggggtgcccaccactccggtgttggttccgcccttgccggtcttgacatgtccatgcccggtgacatcaccttcgactccgctacctccttctggggcaccaaccttaccattgccgtccttaacggtactgtcccccaatggcgtgttgacgacatggccgtccgtatcatggccgcctactacaaggtcggtcgtgaccgtctttaccaaccccccaacttctcctcctggacccgtgacgagtacggtttcaagtacttctacccccaagagggtccctacgagaaggttaaccacttcgtcaacgtccaacgtaaccactccgaggtcatccgtaagttgggtgccgactccaccgtccttttgaagaacaacaacgccttgcccttgaccggtaaggagcgtaaggtcgccatccttggtgaggacgccggttccaactcttacggtgccaacggttgctccgaccgtggttgcgacaacggtactcttgctatggcctggggttccggtactgccgagttcccctaccttgtcacccccgagcaagccatccaagccgaggtcttgaagcacaagggttccgtctacgccatcaccgacaactgggccttgtcccaagtcgagactcttgccaagcaagcctctgtctcccttgttttcgtcaactccgacgccggtgagggttacatctccgttgacggtaacgagggtgaccgtaacaaccttaccctttggaagaacggtgacaaccttatcaaggccgctgccaacaactgcaacaacaccatcgtcgtcatccactccgtcggtcccgtccttgttgacgagtggtacgaccaccccaacgtcaccgccatcctttgggccggtttgcccggtcaagagtccggtaactcccttgccgacgtcctttacggtcgtgtcaaccccggtgccaagtcccccttcacctggggtaagacccgtgaggcttacggtgactaccttgtccgtgagcttaacaacggtaacggtgccccccaagacgacttctccgagggtgttttcatcgactaccgtggtttcgacaagcgtaacgagactcccatctacgagttcggtcacggtttgtcctacaccaccttcaactactccggtctccacatccaagtccttaacgcctcctccaacgcccaagtcgccaccgagactggtgccgcccctaccttcggtcaagtcggtaacgcctccgactacgtctaccccgagggtcttacccgtatctccaagttcatctacccctggcttaactctaccgacttgaaggcttcctccggtgacccctactacggtgttgacaccgccgagcacgtccccgagggtgccaccgacggttccccccaacccgtccttcccgctggtggtggttccggtggtaaccctcgtctttacgacgagcttatccgtgtctccgtcaccgtcaagaacaccggtcgtgtcgccggtgacgccgtcccccaactttacgtttcccttggtggtcccaacgagcccaaggtcgtccttcgtaagttcgaccgtcttaccttgaagccctccgaggagactgtctggaccaccacccttacccgtcgtgacctttccaactgggacgtcgccgcccaagactgggtcatcacctcctaccccaagaaggtccacgtcggttcctcttcccgtcaacttccccttcacgccgcccttcccaaggtccaatgataatctagagctcctgggcctcatgggccttccgctcactgcc

SEQ ID NO: 3 - DNA, 8515 bp, Artificial Sequence
  Description of Artificial Sequence Synthetic pSL6AaBGL1 polynucleotide
cgtacgatttaaatgcggccgcttcggctgcggcgagcgggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaagagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggcctcgtcgagcaagacgtttcccgttgaatatggctcataacaccccttgtattactgtttatgtaagcagacagttttattgttcatttaaatgcggccgcgtacggcggcttcgatagcttcagcctccttaggagcattcaaaccataacgaaggagaagggaagcagataaaattgtaccaacaggattaacaatgcccttgccagcgatatcgggagcgctaccgtgaatgggctcaaccaaacaatgaaccttttcttctgattttcctaccacaccggaaagggaggcagaaggcaaaaggcccaagctaccaggaatgacagaagcctcatctgaaataatgtcaccaaacaagttgtcagtcaaaacaacaccgttaagtgtacgagggctcttgaccaaaagcatggctgcggagtcaatgagctggttttttaaggtaaggtgaggatattcctccttaaaaatcttagctacagtcttgcgccaaagacgagaagttgccaaaacattagctttgtcgagtaatgtgacgggagcaggagggttggaagtttcagctaaccaagcagccaaacgagcaatacgagaaacttcttccaaactgtaaggccaagtgtccatagcataacccgatccgttgtcctcagtgcgctcaccaaagtaacaacctccagtaagttctcgtacaacacaaaaatcgacaccttcaacgatttcaggcttcaaagggctgtacttgactaaagacttgctggcaaagttgcaaggtcgaaggttggcccaaacacccatactcttacgaagcttcaataaaccttgctcaggacgacaattggggttggtccattcaggaccaccaacggcacccaaaagaacaccgtcagcttccaaacaagccttcacagtctcgtcagtcaaaggggttccataggcatcaatagaggcacctccaatcttgtgttcttcaaactcgagttttaactcaggtcgcttcttctcaacgactttcaaaacctccaaggcagaagcaacaatttcagggccaatatggtctcctggtaagacgacgattttctttgcacacatgttgttgaagaagttttgttgtgaaatggtttcgtgaaagtttcagaccctaccgcaaaaatgcctggtttcgggaaactcaacactgttgcactttttatactacagattgggatatcgataatattgcgtaaaaaatcctttttttaaaaagcttgtttacagtaacgtaaatgaccagaaatcagatgaaaatcacaagaaagcaaataattcacgttaaatcctgatatgtttgattttgtgatgaaatcatggatgttcataggaattgttgaaattgcgcttttttaacgaaatatacaagtatcctggagcttacttaattaattaatgaatctttgttcctaggcccgggctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattttgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagatttctctttagttctttgcaagaaggtagagataaagacactttttcaaacatgaagttgtcctggcttgaggccgctgcccttaccgccgcctccgtcgtttccgccgacgagttggccttctccccccccttctacccctccccctgggccaacggtcaaggtgagtgggccgaggcttaccaacgtgccgtcgccattgtctcccaaatgacccttgacgagaaggtcaaccttaccaccggtactggttgggagcttgagaagtgcgtcggtcaaaccggtggtgtcccccgtcttaacatcggtggtatgtgccttcaagactccccccttggtatccgtgactccgactacaactccgccttccctgccggtgtcaacgtcgccgccacctgggacaagaaccttgcctaccttcgtggtcaagctatgggtcaagagttctccgacaagggtatcgacgtccaacttggtcctgccgccggtccccttggtcgttcccccgacggtggtcgtaactgggagggtttctcccccgaccccgcccttaccggtgtccttttcgccgagactatcaagggtatccaagacgctggtgtcgtcgccaccgccaagcactacatccttaacgagcaagagcacttccgtcaagtcgccgaggccgctggttacggtttcaacatctccgacaccatctcttccaacgttgacgacaagaccatccacgagatgtacctttggcccttcgccgacgccgtccgtgccggtgtcggtgccatcatgtgctcctacaaccaaatcaacaactcctacggttgccaaaactcctacacccttaacaagttgttgaaggccgagcttggtttccaaggtttcgtcatgtccgactggggtgcccaccactccggtgttggttccgcccttgccggtcttgacatgtccatgcccggtgacatcaccttcgactccgctacctccttctggggcaccaaccttaccattgccgtccttaacggtactgtcccccaatggcgtgttgacgacatggccgtccgtatcatggccgcctactacaaggtcggtcgtgaccgtctttaccaaccccccaacttctcctcctggacccgtgacgagtacggtttcaagtacttctacccccaagagggtccctacgagaaggttaaccacttcgtcaacgtccaacgtaaccactccgaggtcatccgtaagttgggtgccgactccaccgtccttttgaagaacaacaacgccttgcccttgaccggtaaggagcgtaaggtcgccatccttggtgaggacgccggttccaactcttacggtgccaacggttgctccgaccgtggttgcgacaacggtactcttgctatggcctggggttccggtactgccgagttcccctaccttgtcacccccgagcaagccatccaagccgaggtcttgaagcacaagggttccgtctacgccatcaccgacaactgggccttgtcccaagtcgagactcttgccaagcaagcctctgtctcccttgttttcgtcaactccgacgccggtgagggttacatctccgttgacggtaacgagggtgaccgtaacaaccttaccctttggaagaacggtgacaaccttatcaaggccgctgccaacaactgcaacaacaccatcgtcgtcatccactccgtcggtcccgtccttgttgacgagtggtacgaccaccccaacgtcaccgccatcctttgggccggtttgcccggtcaagagtccggtaactcccttgccgacgtcctttacggtcgtgtcaaccccggtgccaagtcccccttcacctggggtaagacccgtgaggcttacggtgactaccttgtccgtgagcttaacaacggtaacggtgccccccaagacgacttctccgagggtgttttcatcgactaccgtggtttcgacaagcgtaacgagactcccatctacgagttcggtcacggtttgtcctacaccaccttcaactactccggtctccacatccaagtccttaacgcctcctccaacgcccaagtcgccaccgagactggtgccgcccctaccttcggtcaagtcggtaacgcctccgactacgtctaccccgagggtcttacccgtatctccaagttcatctacccctggcttaactctaccgacttgaaggcttcctccggtgacccctactacggtgttgacaccgccgagcacgtccccgagggtgccaccgacggttccccccaacccgtccttcccgctggtggtggttccggtggtaaccctcgtctttacgacgagcttatccgtgtctccgtcaccgtcaagaacaccggtcgtgtcgccggtgacgccgtcccccaactttacgtttcccttggtggtcccaacgagcccaaggtcgtccttcgtaagttcgaccgtcttaccttgaagccctccgaggagactgtctggaccaccacccttacccgtcgtgacctttccaactgggacgtcgccgcccaagactgggtcatcacctcctaccccaagaaggtccacgtcggttcctcttcccgtcaacttccccttcacgccgcccttcccaaggtccaatgataatctagagtcgacctgcaggcatgcaagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgagaaagatgtctatgtagctgaaaataaaatgacgtcacaagacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagcccgggcactagtgaattcgagtatgtgtacgagttgtctttaaacccacagaggtagaatgtatatataaaattaataagctaagtgtaatacttaaaaaatacattaattggaactcgtatcctaccatttacaatgttcatccaattttttcagattgtactgtaaatagcgtttgaaaacaccaaattttagaagctaatcactctcatcataatcgtctacatcctcatcgttatcgacgataaaagaatcatcttgcatgctgggttcatccatgctatcaaacgagggatcaacgtaaataggtgttttcactgtagccgctgctcttctggttggcctctttctaatcggagaatctgaatcttctggtggctctgcgttagtcgaactagcttttggagttgaactactacctggaataataaaatcatcatcgtcatcttcaggtgattgtttctttaccgagcttgcttttttccctttattcttcgcagaagccttcgtggatgttatggtggaaggtttcaaactgctaggcaacaaatcatcttcatcgtctgaagaaaatatggtagtagcaactggtttattagtctttcttcctcttccagacgccgaggctgctatttttttgacgggttttttactacctgcgtcttcagagtcaacagattgacttctttttcttgattttccactatcactgctatccaatcccgggctcttagatatgcgattttcttcaactgataagccatgagagttatcctctgtcttgacaatgtttatgtcagatgatttctcaggttctttcgacgctgcgaactcaagtaaagtttgttgctttcgatttgttgtagatggtttggattcgctgctagcttcttttttaacagcagtacttgaggaggatccggcaatagccctgggtttcctagtaccagtggatttacctcgaggcttctttttcgttcgatttacaaaatctcttgaggattgctcttcttctaacatttctctctgaatatcatccataaccttattccaagcatgctcaaatgcatccaaatcatgaagccacaattctttaggagtttttttaatcaaagcatccagttcggccattacttcgtcctttttcttgagaagttccacataccgttcataggtcaaagaccataaaggcattgaaagaaggtaattgtaggcatctgaatcctcgtcttgcgaaacatcaccagattgttcttcttcagcaagagcattttcaacttctaaatcaaccaaatgccctttctttggtttactgataggttgaaacttcttttccttcagctccacaatgagatcctttttcttcttttttgaaactacaagctccccctctataatcatatgaataaaccgcgcttgatttgaaaatctatcaaaccttttttccaattcattaaccatatgctctttacgtctctggtatgtccttaaacgtacttcgtaaaactcggtcaaaatatcttcaacactgtcatacttcttgatccgtccagatgcatcaaaagcaatcatattactcgttgcttgagtacgcgacagtttaaacttaacttccaaggattcatttaatgcttctttcatgccagcttcggtaagcgtgacattaaagtgaacatttccttcaccgtgatggctttcatagtccacgatgaatttacgaattttttccgtaccaacaagaccagcctccagatactccttcatt

SEQ ID NO: 4 - protein, 876 aa, Artificial Sequence
  Description of Artificial Sequence Synthetic P3AaBGL1 polypeptide
MKITAVIALLFSLAAASPIPVADPGVVSVSLKKRADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 5 - DNA, 8563 bp, Artificial Sequence
  Description of Artificial Sequence Synthetic pSL6P3AaBGL1 polynucleotide
ctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaagagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggcctcgtcgagcaagacgtttcccgttgaatatggctcataacaccccttgtattactgtttatgtaagcagacagttttattgttcatttaaatgcggccgcgtacggcggcttcgatagcttcagcctccttaggagcattcaaaccataacgaaggagaagggaagcagataaaattgtaccaacaggattaacaatgcccttgccagcgatatcgggagcgctaccgtgaatgggctcaaccaaacaatgaaccttttcttctgattttcctaccacaccggaaagggaggcagaaggcaaaaggcccaagctaccaggaatgacagaagcctcatctgaaataatgtcaccaaacaagttgtcagtcaaaacaacaccgttaagtgtacgagggctcttgaccaaaagcatggctgcggagtcaatgagctggttttttaaggtaaggtgaggatattcctccttaaaaatcttagctacagtcttgcgccaaagacgagaagttgccaaaacattagctttgtcgagtaatgtgacgggagcaggagggttggaagtttcagctaaccaagcagccaaacgagcaatacgagaaacttcttccaaactgtaaggccaagtgtccatagcataacccgatccgttgtcctcagtgcgctcaccaaagtaacaacctccagtaagttctcgtacaacacaaaaatcgacaccttcaacgatttcaggcttcaaagggctgtacttgactaaagacttgctggcaaagttgcaaggtcgaaggttggcccaaacacccatactcttacgaagcttcaataaaccttgctcaggacgacaattggggttggtccattcaggaccaccaacggcacccaaaagaacaccgtcagcttccaaacaagccttcacagtctcgtcagtcaaaggggttccataggcatcaatagaggcacctccaatcttgtgttcttcaaactcgagttttaactcaggtcgcttcttctcaacgactttcaaaacctccaaggcagaagcaacaatttcagggccaatatggtctcctggtaagacgacgattttctttgcacacatgttgttgaagaagttttgttgtgaaatggtttcgtgaaagtttcagaccctaccgcaaaaatgcctggtttcgggaaactcaacactgttgcactttttatactacagattgggatatcgataatattgcgtaaaaaatcctttttttaaaaagcttgtttacagtaacgtaaatgaccagaaatcagatgaaaatcacaagaaagcaaataattcacgttaaatcctgatatgtttgattttgtgatgaaatcatggatgttcataggaattgttgaaattgcgcttttttaacgaaatatacaagtatcctggagcttacttaattaattaatgaatctttgttcctaggcccgggctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattttgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagatttctctttagttctttgcaagaaggtagagataaagacactttttcaaacatgaagatcaccgctgtcattgcccttttattctcacttgctgctgcctcacctattccagttgccgatcctggtgtggtttcagttagccttaagaagcgtgccgacgagttggccttctccccccccttctacccctccccctgggccaacggtcaaggtgagtgggccgaggcttaccaacgtgccgtcgccattgtctcccaaatgacccttgacgagaaggtcaaccttaccaccggtactggttgggagcttgagaagtgcgtcggtcaaaccggtggtgtcccccgtcttaacatcggtggtatgtgccttcaagactccccccttggtatccgtgactccgactacaactccgccttccctgccggtgtcaacgtcgccgccacctgggacaagaaccttgcctaccttcgtggtcaagctatgggtcaagagttctccgacaagggtatcgacgtccaacttggtcctgccgccggtccccttggtcgttcccccgacggtggtcgtaactgggagggtttctcccccgaccccgcccttaccggtgtccttttcgccgagactatcaagggtatccaagacgctggtgtcgtcgccaccgccaagcactacatccttaacgagcaagagcacttccgtcaagtcgccgaggccgctggttacggtttcaacatctccgacaccatctcttccaacgttgacgacaagaccatccacgagatgtacctttggcccttcgccgacgccgtccgtgccggtgtcggtgccatcatgtgctcctacaaccaaatcaacaactcctacggttgccaaaactcctacacccttaacaagttgttgaaggccgagcttggtttccaaggtttcgtcatgtccgactggggtgcccaccactccggtgttggttccgcccttgccggtcttgacatgtccatgcccggtgacatcaccttcgactccgctacctccttctggggcaccaaccttaccattgccgtccttaacggtactgtcccccaatggcgtgttgacgacatggccgtccgtatcatggccgcctactacaaggtcggtcgtgaccgtctttaccaaccccccaacttctcctcctggacccgtgacgagtacggtttcaagtacttctacccccaagagggtccctacgagaaggttaaccacttcgtcaacgtccaacgtaaccactccgaggtcatccgtaagttgggtgccgactccaccgtccttttgaagaacaacaacgccttgcccttgaccggtaaggagcgtaaggtcgccatccttggtgaggacgccggttccaactcttacggtgccaacggttgctccgaccgtggttgcgacaacggtactcttgctatggcctggggttccggtactgccgagttcccctaccttgtcacccccgagcaagccatccaagccgaggtcttgaagcacaagggttccgtctacgccatcaccgacaactgggccttgtcccaagtcgagactcttgccaagcaagcctctgtctcccttgttttcgtcaactccgacgccggtgagggttacatctccgttgacggtaacgagggtgaccgtaacaaccttaccctttggaagaacggtgacaaccttatcaaggccgctgccaacaactgcaacaacaccatcgtcgtcatccactccgtcggtcccgtccttgttgacgagtggtacgaccaccccaacgtcaccgccatcctttgggccggtttgcccggtcaagagtccggtaactcccttgccgacgtcctttacggtcgtgtcaaccccggtgccaagtcccccttcacctggggtaagacccgtgaggcttacggtgactaccttgtccgtgagcttaacaacggtaacggtgccccccaagacgacttctccgagggtgttttcatcgactaccgtggtttcgacaagcgtaacgagactcccatctacgagttcggtcacggtttgtcctacaccaccttcaactactccggtctccacatccaagtccttaacgcctcctccaacgcccaagtcgccaccgagactggtgccgcccctaccttcggtcaagtcggtaacgcctccgactacgtctaccccgagggtcttacccgtatctccaagttcatctacccctggcttaactctaccgacttgaaggcttcctccggtgacccctactacggtgttgacaccgccgagcacgtccccgagggtgccaccgacggttccccccaacccgtccttcccgctggtggtggttccggtggtaaccctcgtctttacgacgagcttatccgtgtctccgtcaccgtcaagaacaccggtcgtgtcgccggtgacgccgtcccccaactttacgtttcccttggtggtcccaacgagcccaaggtcgtccttcgtaagttcgaccgtcttaccttgaagccctccgaggagactgtctggaccaccacccttacccgtcgtgacctttccaactgggacgtcgccgcccaagactgggtcatcacctcctaccccaagaaggtccacgtcggttcctcttcccgtcaacttccccttcacgccgcccttcccaaggtccaatgataatctagagtcgacctgcaggcatgcaagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgagaaagatgtctatgtagctgaaaataaaatgacgtcacaagacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagcccgggcactagtgaattcgagtatgtgtacgagttgtctttaaacccacagaggtagaatgtatatataaaattaataagctaagtgtaatacttaaaaaatacattaattggaactcgtatcctaccatttacaatgttcatccaattttttcagattgtactgtaaatagcgtttgaaaacaccaaattttagaagctaatcactctcatcataatcgtctacatcctcatcgttatcgacgataaaagaatcatcttgcatgctgggttcatccatgctatcaaacgagggatcaacgtaaataggtgttttcactgtagccgctgctcttctggttggcctctttctaatcggagaatctgaatcttctggtggctctgcgttagtcgaactagcttttggagttgaactactacctggaataataaaatcatcatcgtcatcttcaggtgattgtttctttaccgagcttgcttttttccctttattcttcgcagaagccttcgtggatgttatggtggaaggtttcaaactgctaggcaacaaatcatcttcatcgtctgaagaaaatatggtagtagcaactggtttattagtctttcttcctcttccagacgccgaggctgctatttttttgacgggttttttactacctgcgtcttcagagtcaacagattgacttctttttcttgattttccactatcactgctatccaatcccgggctcttagatatgcgattttcttcaactgataagccatgagagttatcctctgtcttgacaatgtttatgtcagatgatttctcaggttctttcgacgctgcgaactcaagtaaagtttgttgctttcgatttgttgtagatggtttggattcgctgctagcttcttttttaacagcagtacttgaggaggatccggcaatagccctgggtttcctagtaccagtggatttacctcgaggcttctttttcgttcgatttacaaaatctcttgaggattgctcttcttctaacatttctctctgaatatcatccataaccttattccaagcatgctcaaatgcatccaaatcatgaagccacaattctttaggagtttttttaatcaaagcatccagttcggccattacttcgtcctttttcttgagaagttccacataccgttcataggtcaaagaccataaaggcattgaaagaaggtaattgtaggcatctgaatcctcgtcttgcgaaacatcaccagattgttcttcttcagcaagagcattttcaacttctaaatcaaccaaatgccctttctttggtttactgataggttgaaacttcttttccttcagctccacaatgagatcctttttcttcttttttgaaactacaagctccccctctataatcatatgaataaaccgcgcttgatttgaaaatctatcaaaccttttttccaattcattaaccatatgctctttacgtctctggtatgtccttaaacgtacttcgtaaaactcggtcaaaatatcttcaacactgtcatacttcttgatccgtccagatgcatcaaaagcaatcatattactcgttgcttgagtacgcgacagtttaaacttaacttccaaggattcatttaatgcttctttcatgccagcttcggtaagcgtgacattaaagtgaacatttccttcaccgtgatggctttcatagtccacgatgaatttacgaattttttccgtaccaacaagaccagcctccagatactccttcattcgtacgatttaaatgcggccgcttcggctgcggcgagcgggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatg

SEQ ID NO: 6 - DNA, 29 bp, Artificial Sequence
  Description of Artificial Sequence Synthetic mut fw2 primer
ctctttagttctttgcaagaaggtagaga

SEQ ID NO: 7 - DNA, 21 bp, Artificial Sequence
  Description of Artificial Sequence Synthetic mut rv3 primer
atgcctgcaggtcgactctag

SEQ ID NO: 8 - DNA, 21 bp, Artificial Sequence
  Description of Artificial Sequence Synthetic leu fw1 primer
cctcgtcgagcaagacgtttc

SEQ ID NO: 9 - DNA, 33 bp, Artificial Sequence
  Description of Artificial Sequence Synthetic leu rv1 primer
catgtttgaaaaagtgtctttatctctaccttc

SEQ ID NO: 10 - DNA, 19 bp, Artificial Sequence
  Description of Artificial Sequence Synthetic top fw3 primer
cgcccttcccaaggtccaa

SEQ ID NO: 11 - DNA, 23 bp, Artificial Sequence
  Description of Artificial Sequence Synthetic top rv1 primer
ctgtggataaccgtattaccgcc

SEQ ID NO: 12 - DNA, 23 bp, Artificial Sequence
  Description of Artificial Sequence Synthetic leu fw3 primer
cggcttcgatagcttcagcctcc

SEQ ID NO: 13 - DNA, 25 bp, Artificial Sequence
  Description of Artificial Sequence Synthetic top rv5 primer
ttcgcaagacgaggattcagatgcc

SEQ ID NO: 14 - DNA, 30 bp, Artificial Sequence
  Description of Artificial Sequence Synthetic leu1 primer
cctcacctcccaaatcgcgagtataaagac

SEQ ID NO: 15 - DNA, 30 bp, Artificial Sequence
  Description of Artificial Sequence Synthetic top2 primer
agctagcagcgaatccaaaccatctacaac

SEQ ID NO: 16 - DNA, 43 bp, Artificial Sequence
  Description of Artificial Sequence Synthetic P3AaBGL1 fw primer
ggtttcagttagccttaagaagcgtgccgacgagttggccttc

SEQ ID NO: 17 - DNA, 27 bp, Artificial Sequence
  Description of Artificial Sequence Synthetic P3AaBGL1 rv primer
ctgcaggtcgactctagattatcattg

SEQ ID NO: 18 - DNA, 32 bp, Artificial Sequence
  Description of Artificial Sequence Synthetic G298X rv primer
gaaccaacaccggagtggtgggcsnnccagtc

SEQ ID NO: 19 - DNA, 33 bp, Artificial Sequence
  Description of Artificial Sequence Synthetic G298X fw primer
caaggtttcgtcatgtccgactggnnsgcccac

SEQ ID NO: 20 - DNA, 33 bp, Artificial Sequence
  Description of Artificial Sequence Synthetic V519X rv primer
gtaaccctcaccggcgtcggagttsnngaaaac

SEQ ID NO: 21 - DNA, 33 bp, Artificial Sequence
  Description of Artificial Sequence Synthetic V519X fw primer
caagcctctgtctcccttgttttcnnsaactcc

SEQ ID NO: 22 - DNA, 30 bp, Artificial Sequence
  Description of Artificial Sequence Synthetic Q142X rv primer
ggagaactcttgacccatagcsnnaccacg

SEQ ID NO: 23 - DNA, 33 bp, Artificial Sequence
  Description of Artificial Sequence Synthetic Q142X fw primer
aagaaccttgcctaccttcgtggtnnsgctatg

SEQ ID NO: 24 - DNA, 27 bp, Artificial Sequence
  Description of Artificial Sequence Synthetic Q142X rv primer
gtaggggaactcggcagtsnnggaacc

SEQ ID NO: 25 - DNA, 24 bp, Artificial Sequence
  Description of Artificial Sequence Synthetic G468X fw primer
atggcctggggttccnnsactgcc

SEQ ID NO: 26 - DNA, 24 bp, Artificial Sequence
  Description of Artificial Sequence Synthetic Q156L rv primer
gcggcaggaccaagtaggacgtcg

SEQ ID NO: 27 - DNA, 27 bp, Artificial Sequence
  Description of Artificial Sequence Synthetic Q156L fw primer
caagggtatcgacgtcctacttggtcc

SEQ ID NO: 28 - DNA, 26 bp, Artificial Sequence
  Description of Artificial Sequence Synthetic I260F rv primer
ggttgtaggagcacatgaaggcaccg

SEQ ID NO: 29 - DNA, 24 bp, Artificial Sequence
  Description of Artificial Sequence Synthetic I260F fw primer
tgccggtgtcggtgccttcatgtg

SEQ ID NO: 30 - DNA, 25 bp, Artificial Sequence
  Description of Artificial Sequence Synthetic N458S rv primer
gccatagcaagagtaccgctgtcgc

SEQ ID NO: 31 - DNA, 25 bp, Artificial Sequence
  Description of Artificial Sequence Synthetic N458S fw primer
gaccgtggttgcgacagcggtactc

SEQ ID NO: 32 - DNA, 29 bp, Artificial Sequence
  Description of Artificial Sequence Synthetic G468X fw primer
ccgttaccgttgttaagctcaaggacaag

SEQ ID NO: 33 - DNA, 31 bp, Artificial Sequence
  Description of Artificial Sequence Synthetic G468X fw primer
gcttacggtgactaccttgtccttgagctta

(19) United States
(12) Patent Application Publication
(10) Pub. No.: US 2014/0186897 A1
(43) Pub. Date: Jul. 3, 2014

(54) TRANSFORMANT OF YEAST OF GENUS SCHIZOSACCHAROMYCES, METHOD FOR PRODUCING SAME, METHOD FOR PRODUCING ß-GLUCOSIDASE, AND CELLULOSE DEGRADATION METHOD

(71) Applicant: ASAHI GLASS COMPANY, LIMITED, Tokyo (JP)

(72) Inventors: Katsunori OKADA, Tokyo (JP); Hideki Tohda, Tokyo (JP)

(73) Assignee: ASAHI GLASS COMPANY, LIMITED, Tokyo (JP)

(21) Appl. No.: 14/031,575

(22) Filed: Sep. 19, 2013

Related U.S. Application Data
(63) Continuation of application No. PCT/JP2012/057055, filed on Mar. 19, 2012.

(30) Foreign Application Priority Data
Mar. 24, 2011 (JP) 2011-066539

(51) Int. Cl. C12P 19/14; C12N 15/81; C12P 19/02; C12N 9/42

(52) U.S. Cl. CPC C12P 19/14; C12N 9/2445; C12N 15/815; C12P 19/02
USPC 435/99; 435/471; 435/209; 435/254.2

(57) ABSTRACT

To provide a transformant of a yeast of the genus Schizosaccharomyces which can produce a modified-type β-glucosidase with improved glucosidase activity, a method for producing such a transformant, a method for producing the modified-type β-glucosidase, and a cellulose degradation method using the modified-type β-glucosidase. A transformant of a yeast of the genus Schizosaccharomyces characterized by having a structural gene sequence comprising a region encoding a modified-type β-glucosidase which is obtained by introducing at least one mutation selected from the group consisting of specific mutations into a β-glucosidase derived from a filamentous fungus, and a promoter sequence and a terminator sequence for expressing the structural gene in a chromosome, or alternatively by having the sequences as an extrachromosomal gene, and is characterized in that the modified-type β-glucosidase has higher thermostability or higher resistance to glucose inhibition than a β-glucosidase which does not have the specific mutations.

### TECHNICAL FIELD

The present invention relates to a transformant of a yeast of the genus Schizosaccharomyces, a method for producing the transformant, a method for producing β-glucosidase by using the transformant, and a cellulose degradation method using the transformant. Specifically, the present invention relates to a transformant of a yeast of the genus Schizosaccharomyces which can produce a modified-type β-glucosidase having higher thermostability or higher resistance to glucose inhibition than a wild-type β-glucosidase, and a method for producing such a transformant.

### BACKGROUND ART

To produce biomass fuels from a cellulosic biomass such as a wood, rice straw, rice husk and weed including a sugar as a fermentation feedstock, and a bioethanol, etc., it is required to degrade the main structural component of a plant cell wall, cellulose. To degrade cellulose, an acid saccharification method such as a concentrated sulfuric acid saccharification method or a dilute sulfuric acid saccharification method, an enzymatic saccharification method, etc. may be employed. Because of recent development in biotechnology, research and development of an enzymatic saccharification method are actively carried out. In the enzymatic saccharification of cellulose, a group of enzymes generally known as cellulases are utilized. Firstly, an endoglucanase (EG), which has an activity to cleave cellulose chains at random, degrades an amorphous region of cellulose to expose terminal glucose residues. The exposed glucose residues are degraded by a cellobiohydrolase (CBH) to release cellobiose. Thereafter, the released cellobiose is degraded by β-glucosidase (BGL) to release glucose.

For the saccharification of cellulose, filamentous fungi of the genus Aspergillus and the genus Trichoderma are widely used, since they can produce various cellulases and hemicellulases which are required for decomposing and saccharifying a crystalline cellulose, and they can secrete a large amount of such enzymes to their extracellular environment.

Further, it has been tried to express such cellulases of filamentous fungi in a heterologous microorganism. Non-patent document 1 discloses that a budding yeast Saccharomyces cerevisiae was transformed with a gene encoding β-glucosidase 1 (BGL 1) of Aspergillus aculeatus to obtain a transformant, and the obtained transformant expressed such an enzyme.

On the other hand, the genetic analysis of a yeast of the genus Schizosaccharomyces is more advanced than that of a filamentous fungus of the genus Aspergillus or the genus Trichoderma, and the yeast has a lot of advantages like availability of various useful mutant strains and gene transfer vectors, and its suitability for industrial large-scale production of a protein. However, the yeast of the genus Schizosaccharomyces does not have endogenous β-glucosidase gene, whereby it cannot utilize cellobiose.

Further, development of a highly functional β-glucosidase has been desired. For example, in the enzymatic saccharification method, as the enzymatic hydrolysis of cellulose proceeds, glucose accumulates in the reaction system and the accumulated glucose inhibits β-glucosidase, whereby accumulation of cellobiose proceeds. Further, there is a problem such that the complete degradation of cellulose may not be achieved since the accumulated cellobiose inhibits endoglucanase and cellobiohydrolase. Accordingly, development of a β-glucosidase having higher resistance to glucose inhibition (i.e. less susceptible to glucose inhibition) has been desired.

Further, in general, there is a tendency for an enzymatic reaction such that the higher the reaction temperature, the higher the reaction efficiency. Therefore, by increasing the temperature during cellulose degradation reaction, the efficiency of the cellulose degradation reaction can be increased. Furthermore, as the thermostability increases, it becomes possible to continue enzymatic reaction for a longer period of time. Accordingly, development of a β-glucosidase having higher thermostability has been desired.

Random mutagenesis of an enzyme is one of important techniques for improving the function of the enzyme. By using an error-prone PCR (polymerase chain reaction) method (Non-Patent Document 2), etc., a single-nucleotide substitution is introduced into a target gene in a random manner. By using saturation mutagenesis (Non-Patent Document 2), a codon at a specific site is modified in a random manner. By in vitro recombination (Non-Patent Document 2), several mutant genes are recombined to obtain a combination of specific mutation points. By means of the above-described random mutagenesis methods, a random mutation can be introduced into a gene encoding a target enzyme, whereby a mutant enzyme library can be constructed. It is possible to obtain a useful modified enzyme by using a screening method for selecting one having a desired function from the library. Further, useful mutation points can be accumulated by repeating random mutagenesis and screening, whereby functions of the enzyme can be improved further.

However, the improvement of enzyme by random mutagenesis is mostly reported in the cases of E. coli (Patent Document 1) and a cell-free protein synthesis system (Patent Document 2), and is not suitable for the mutagenesis of glycoproteins derived from eukaryotes. Further, Patent Document 3 discloses random mutagenesis in budding yeast which requires step to insert a mutant gene into a vector, step to amplify the mutant-gene inserted vector in E. coli and step to purify the vector, and which is not simple.

### PRIOR ART DOCUMENTS

### Patent Documents

Patent Document 1: Japanese Patent No. 4402446
Patent Document 2: JP-A-2005-245336
Patent Document 3: Japanese Patent No. 4448772

### Non-Patent Documents

Non-Patent Document 1: G. Tanaka, et al., Biosci. Biotechnol. Biochem., (1998) 62(8), p 1615-1618.
Non-Patent Document 2: F. Arnold, et al., “Directed Evolution Library Creation Methods and Protocols”, Methods in Molecular Biology, (2003) Vol. 231.

### DISCLOSURE OF INVENTION

### Technical Problem

Here, the object of the present invention is to provide a transformant of a yeast of the genus Schizosaccharomyces which can produce a modified-type β-glucosidase with improved glucosidase activity, a method for producing such a transformant, a method for producing the modified-type β-glucosidase, and a cellulose degradation method using the modified-type β-glucosidase.

### Solution To Problem

The present inventors have conducted extensive studies to resolve the above-mentioned problems, and as a result, have found that the above-mentioned problems can be resolved by means of a transformant of a yeast the genus Schizosaccharomyces having a structural gene encoding a modified-type β-glucosidase which is obtained by introducing a specific mutation into a structural gene sequence encoding a β-glucosidase derived from a filamentous fungus.

That is, the present invention provides the following [1] to [15].

[1] A transformant of a yeast of the genus Schizosaccharomyces characterized by having a structural gene sequence comprising a region encoding the following modified-type β-glucosidase, and a promoter sequence and a terminator sequence for expressing the structural gene in a chromosome, or alternatively by having the sequences as an extrachromosomal gene.

Modified-type β-glucosidase: Aβ-glucosidase derived from a filamentous fungus having at least one mutation selected from the group consisting of the following mutations 1 to 6, and has higher thermostability or higher resistance to glucose inhibition than a β-glucosidase which does not have the mutations.

Mutation 1; a mutation in which an amino acid corresponds to isoleucine at position 244 of AaBGL1 (BGL1 of Aspergillus aculeatus) is substituted by phenylalanine.

Mutation 2; a mutation in which an amino acid corresponds to glycine at position 282 of AaBGL1 is substituted by phenylalanine.

Mutation 3; a mutation in which an amino acid corresponds to asparagine at position 442 of AaBGL1 is substituted by serine.

Mutation 4; a mutation in which an amino acid corresponds to glycine at position 452 of AaBGL1 is substituted by serine.

Mutation 5; a mutation in which an amino acid corresponds to valine at position 503 of AaBGL1 is substituted by alanine.

Mutation 6; a mutation in which an amino acid corresponds to arginine at position 613 of AaBGL1 is substituted by leucine.

[2] The transformant of a yeast of the genus Schizosaccharomyces according to [1], wherein the β-glucosidase derived from a filamentous fungus is BGL1.
[3] The transformant of a yeast of the genus Schizosaccharomyces according to [1] or [2], wherein the filamentous fungus is a microorganism of the genus Aspergillus.
[4] The transformant of a yeast of the genus Schizosaccharomyces according to any one of Claims 1 to 3, wherein the β-glucosidase derived from a filamentous fungus is comprised of an amino acid sequence having at least 70% homology with an amino acid sequence of SEQ ID NO: 1, and is a protein has a catalytic activity to hydrolyze a β-D-glucopyranoside bond.
[5] The transformant of a yeast of the genus Schizosaccharomyces according to any one of [1] to [4], wherein the structural gene sequence further comprises a region encoding a signal or a region encoding a tag at the 5′ end side or the 3′ end side of the region encoding the modified-type β-glucosidase.
[6] The transformant of a yeast of the genus Schizosaccharomyces according to any one of [1] to [5], wherein the structural gene sequence further comprises a region encoding a secretion signal at the 5′ end side of the region encoding the modified-type β-glucosidase.
[7] A transformation method for a yeast of the genus Schizosaccharomyces, characterized in that the yeast of the genus Schizosaccharomyces is transformed by using a vector containing a structural gene sequence comprising a region encoding the following modified-type β-glucosidase, and a promoter sequence and a terminator sequence for expressing the structural gene.

Modified-type β-glucosidase: Aβ-glucosidase derived from a filamentous fungus having at least one mutation selected from the group consisting of the following mutations 1 to 6, and has higher thermostability or higher resistance to glucose inhibition than a β-glucosidase which does not have the mutations.

Mutation 1; a mutation in which an amino acid corresponds to isoleucine at position 244 of AaBGL1 (BGL1 of Aspergillus aculeatus) is substituted by phenylalanine.

Mutation 2; a mutation in which an amino acid corresponds to glycine at position 282 of AaBGL1 is substituted by phenylalanine.

Mutation 3; a mutation in which an amino acid corresponds to asparagine at position 442 of AaBGL1 is substituted by serine.

Mutation 4; a mutation in which an amino acid corresponds to glycine at position 452 of AaBGL1 is substituted by serine.

Mutation 5; a mutation in which an amino acid corresponds to valine at position 503 of AaBGL1 is substituted by alanine.

Mutation 6; a mutation in which an amino acid corresponds to arginine at position 613 of AaBGL1 is substituted by leucine.

[8] The transformation method for a yeast of the genus Schizosaccharomyces according to [7], wherein the structural gene sequence further comprises a region encoding a signal or a region encoding a tag at the 5′ end side or the 3′ end side of the region encoding the modified-type β-glucosidase.
[9] The transformation method for a yeast of the genus Schizosaccharomyces according to [7], wherein the structural gene sequence further comprises a region encoding a secretion signal at the 5′ end side of the region encoding the modified-type β-glucosidase.
[10] The transformation method for a yeast of the genus Schizosaccharomyces according to any one of [7] to [9], wherein the vector is integrated into at least one position of a chromosome of the yeast of the genus Schizosaccharomyces.
[11] The transformation method for a yeast of the genus Schizosaccharomyces according to [10], wherein the vector is integrated into a transposon gene Tf2 site of the chromosome.
[12] A method for producing a β-glucosidase, characterized in that the modified-type β-glucosidase is recovered from cells obtained by cultivating the transformant as defined in any one of [1] to [5].
[13] A method for producing a β-glucosidase, characterized in that the modified-type β-glucosidase is recovered from a culture supernatant obtained by cultivating the transformant as defined in [6].
[14] A cellulose degradation method, characterized in that the modified-type β-glucosidase obtained by the production method as defined in [12] or [13] is used.
[15] A cellulose degradation method, characterized in that the transformant as defined in [6] is cultivated in the presence of cellulose.

### Advantageous Effects of Invention

According to the transformation method for a yeast of the genus Schizosaccharomyces of the present invention, it is possible to produce a transformant of a yeast of the genus Schizosaccharomyces which can produce a modified-type β-glucosidase and is improved at least one of thermostability or resistance to glucose inhibition. Further, the modified-type β-glucosidase recovered from the transformant by the method for producing β-glucosidase of the present invention is suitably used for degradation of cellulose.

### BRIEF DESCRIPTION OF DRAWINGS

FIG. 1 shows a phylogenetic tree of β-glucosidases derived from filamentous fungi along with homologies to BGL1 (AaBGL1) encoded by the bgl1 gene of Aspergillus aculeatus.

FIG. 2A shows alignments of the amino acid sequence of AaBGL1 and the amino acid sequences of β-glucosidases derived from other filamentous fungi.

FIG. 2B shows alignments of the amino acid sequence of AaBGL1 and the amino acid sequences of β-glucosidases derived from other filamentous fungi.

FIG. 2C shows alignments of the amino acid sequence of AaBGL1 and the amino acid sequences of β-glucosidases derived from other filamentous fungi.

FIG. 3 shows a structure map of the expression vector pSL6AaBGL1.

FIG. 4 shows a structure map of the expression vector pSL6P3AaBGL1.

FIGS. 5A and 5B show the test results of Example 8 in which the glucose inhibition (FIG. 5A) and the thermostability at a warming temperature of from 50 to 75° C. (FIG. 5B) of the culture supernatants of the respective mutant strains were measured.

### DESCRIPTION OF EMBODIMENTS

β-glucosidase (EC.3.2.1.21) is a generic name of an enzyme which specifically catalyzes hydrolysis of a β-D-glucopyranoside bond. Particularly, it is also called as cellobiase since it degrades cellobiose to glucose, and is widely found in bacteria, filamentous fungi, plants and animals. A plurality of genes encoding β-glucosidase is usually found in each of the species, and for example, existence of bgl1 to bgl7 in a filamentous fungus Aspergillus oryzae has been reported (Soy Protein Research, Japan, Vol. 12, pp. 78-83, 2009; and JP-A-2008-086310).

In the present invention and present specification, “β-glucosidase derived from a filamentous fungus” means a wild-type β-glucosidase intrinsic to a filamentous fungus. The filamentous fungus is, among fungi, an eukaryotic microorganism composed of tubular cells called hyphae. As the filamentous fungus, a fungus of the genus Aspergillus, the genus Trichoderma, the genus Fusarium, the genus Penicillium, the genus Acremonium or the like may, for example, be mentioned. As the filamentous fungus of the genus Aspergillus, Aspergillus nidulans, Aspergillus oryzae, Aspergillus aculeatus, Aspergillus niger, Aspergillus pulverulentus, Aspergillus terreus or the like may, for example, be mentioned.

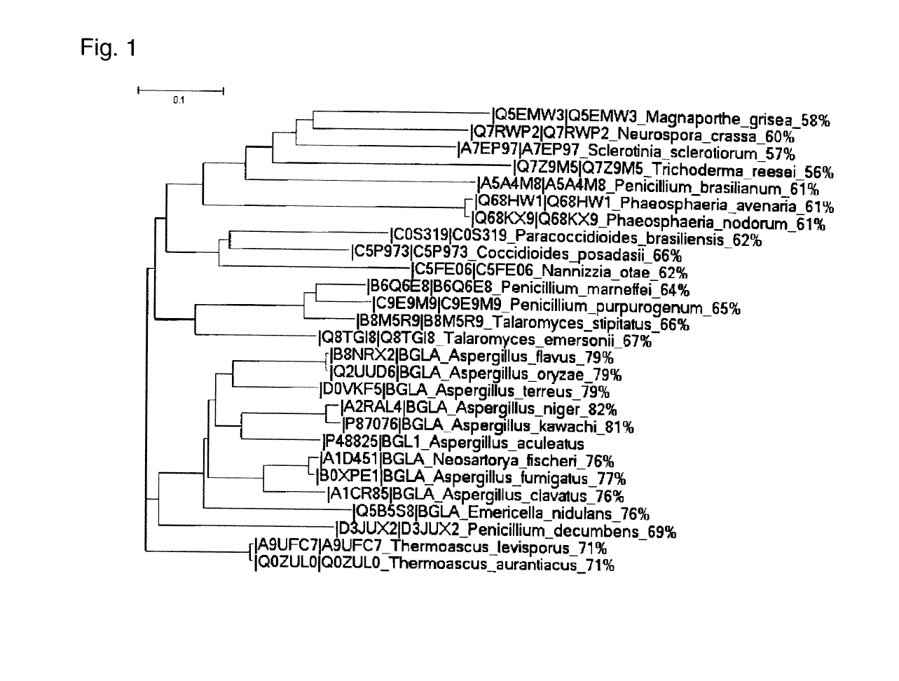

In FIG. 1, a phylogenetic tree of β-glucosidases derived from filamentous fungi is shown along with homologies (%: a value of the terminal of each β-glucosidase) to BGL1 (AaBGL1) encoded by the bgl1 gene of Aspergillus aculeatus. Further, in FIG. 1, the first six characters of each β-glucosidase indicate the accession numbers registered in the UniProtKB/Swiss-Prot Database.

In the present specification and present invention, high resistance to glucose inhibition means that the ratio of the activity to catalyze hydrolysis of a β-D-glucopyranoside bond (hereinafter referred to as glucosidase activity) in the presence of glucose to the glucosidase activity in the absence of glucose (i.e. the relative activity value of the glucosidase activity in the presence of glucose, assuming that the glucosidase activity in the absence of glucose is 100%) is high. Further, high thermostability means that the ratio of the glucosidase activity for the case where heating treatment is carried out to the glucosidase activity for the case where heating treatment is not carried out before enzymatic reaction is high.

### [Transformant]

The transformant of the present invention is characterized by having a structural gene sequence comprising a region encoding the following modified-type β-glucosidase, and a promoter sequence and a terminator sequence for expressing the structural gene in a chromosome, or alternatively by having the sequences as an extrachromosomal gene. The modified-type β-glucosidase is a β-glucosidase derived from a filamentous fungus having at least one mutation selected from the group consisting of the below-described mutations 1 to 6, and has higher thermostability or higher resistance to glucose inhibition than a β-glucosidase which does not have the mutations. The modified-type β-glucosidase can be obtained by introducing a specific amino acid substitution mutation into a β-glucosidase derived from a filamentous fungus.

### (Modified-Type β-Glucosidase)

The modified-type β-glucosidase is an enzyme which has, as compared to a β-glucosidase derived from a filamentous fungus, at least one mutation selected from the group consisting of the following mutations 1 to 6 and has a glucosidase activity. The modified-type β-glucosidase which has mutation 2 or mutation 4 shows higher resistance to glucose inhibition than a β-glucosidase which does not have such mutations. Further, the modified-type β-glucosidase which has mutation 3, mutation 5, or mutation 6 shows higher thermostability than a β-glucosidase which does not have such mutations. The modified-type β-glucosidase which has mutation 1 shows both higher resistance to glucose inhibition and higher thermostability than a β-glucosidase which does not have such mutation.

Mutation 1; a mutation in which an amino acid corresponds to isoleucine at position 244 of AaBGL1 (BGL1 of Aspergillus aculeatus) is substituted by phenylalanine.

Mutation 2; a mutation in which an amino acid corresponds to glycine at position 282 of AaBGL1 is substituted by phenylalanine.

Mutation 3; a mutation in which an amino acid corresponds to asparagine at position 442 of AaBGL1 is substituted by serine.

Mutation 4; a mutation in which an amino acid corresponds to glycine at position 452 of AaBGL1 is substituted by serine.

Mutation 5; a mutation in which an amino acid corresponds to valine at position 503 of AaBGL1 is substituted by alanine.

Mutation 6; a mutation in which an amino acid corresponds to arginine at position 613 of AaBGL1 is substituted by leucine.

Further, it was firstly found by the present inventors that thermostability or resistance to glucose inhibition can be increased by having the above-described mutations 1 to 6. Such a finding was obtained by a random mutagenesis using a yeast of the genus Schizosaccharomyces as a host and a method for recovering an improved protein via functional screening. Specifically, various mutations were introduced into AaBGL1 by random mutagenesis, and thus obtained AaBGL1 was introduced into a yeast of the genus Schizosaccharomyces to construct a mutant-type β-glucosidase library. Thereafter, thus obtained library was screened to select a transformant having improved thermostability or resistance to glucose inhibition, thereby to analyze the nucleotide sequence of a structural gene encoding AaBGL1 of the transformant.

Modified-type β-glucosidase may contain only one type of mutation or at least two types of mutations in combination, among the above-described six types of mutations. For example, a modified-type β-glucosidase which has both mutation 2 and mutation 4 shows higher resistance to glucose inhibition than a β-glucosidase which has only one of them. Further, for example, a modified-type β-glucosidase which has mutation 5 and mutation 6 shows higher thermostability than a modified-type β-glucosidase which has only mutation 5. Further, a modified-type β-glucosidase which has mutation 3, mutation 5 and mutation 6 shows higher thermostability than a modified-type β-glucosidase which has mutation 5 and mutation 6. Further, a modified-type β-glucosidase which has mutation 1, mutation 2, mutation 5 and mutation 6 shows higher thermostability than a modified-type β-glucosidase which has only mutation 1 and mutation 2.

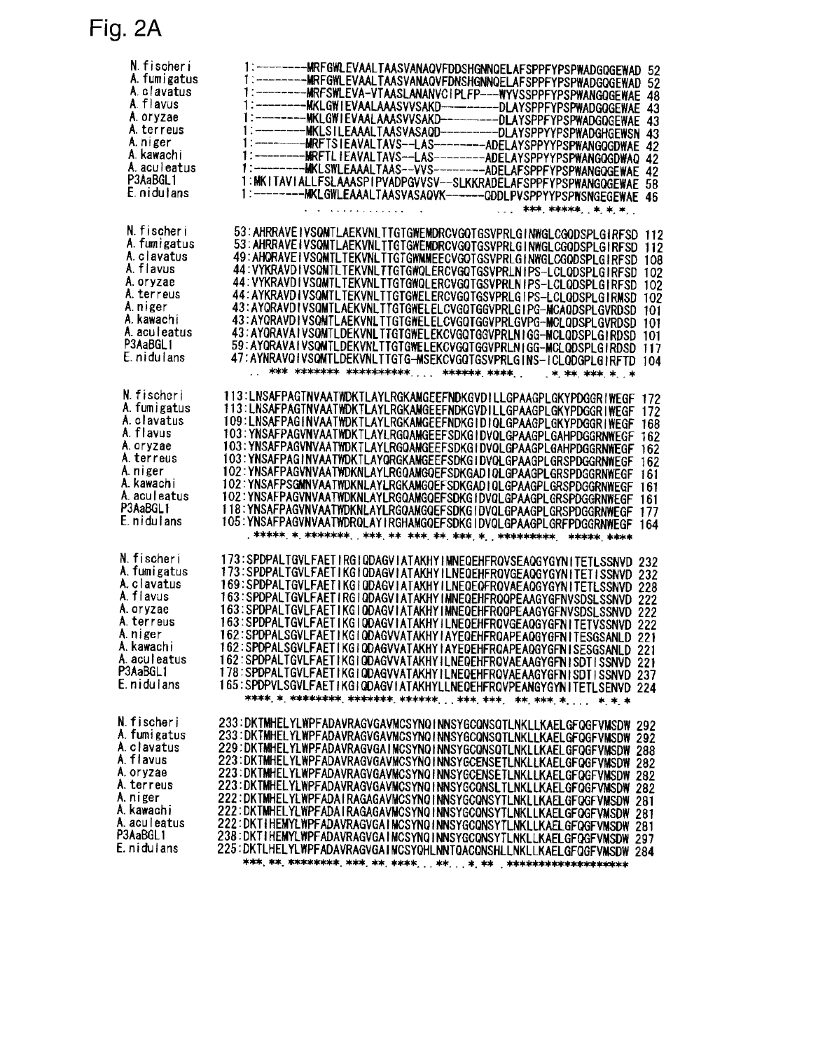

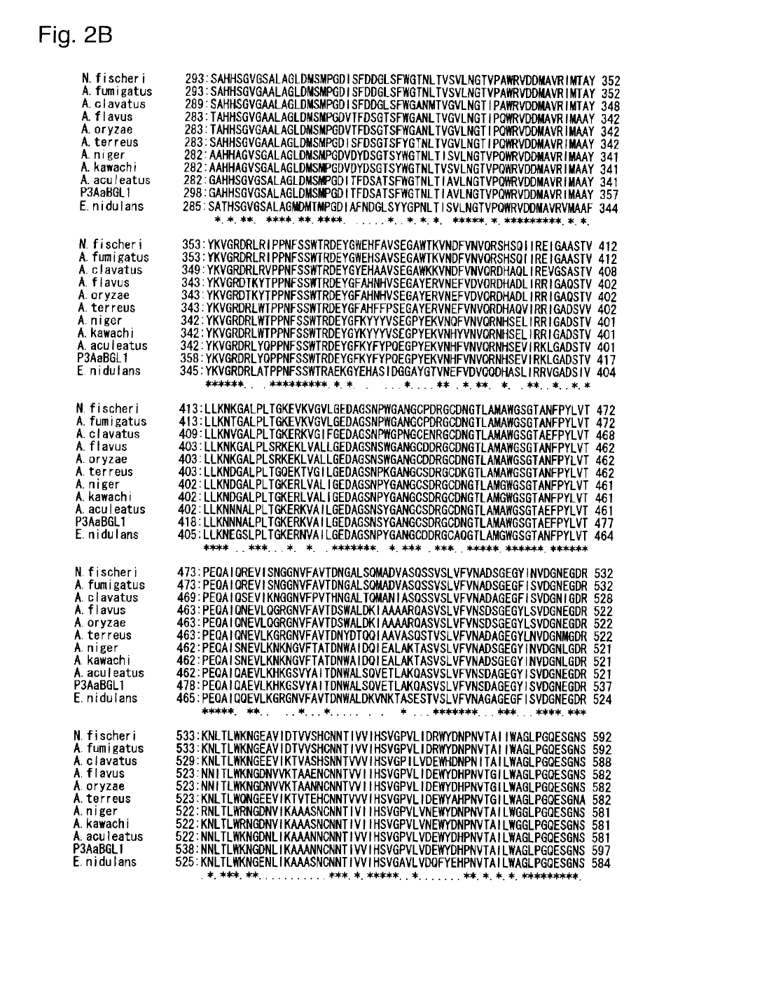

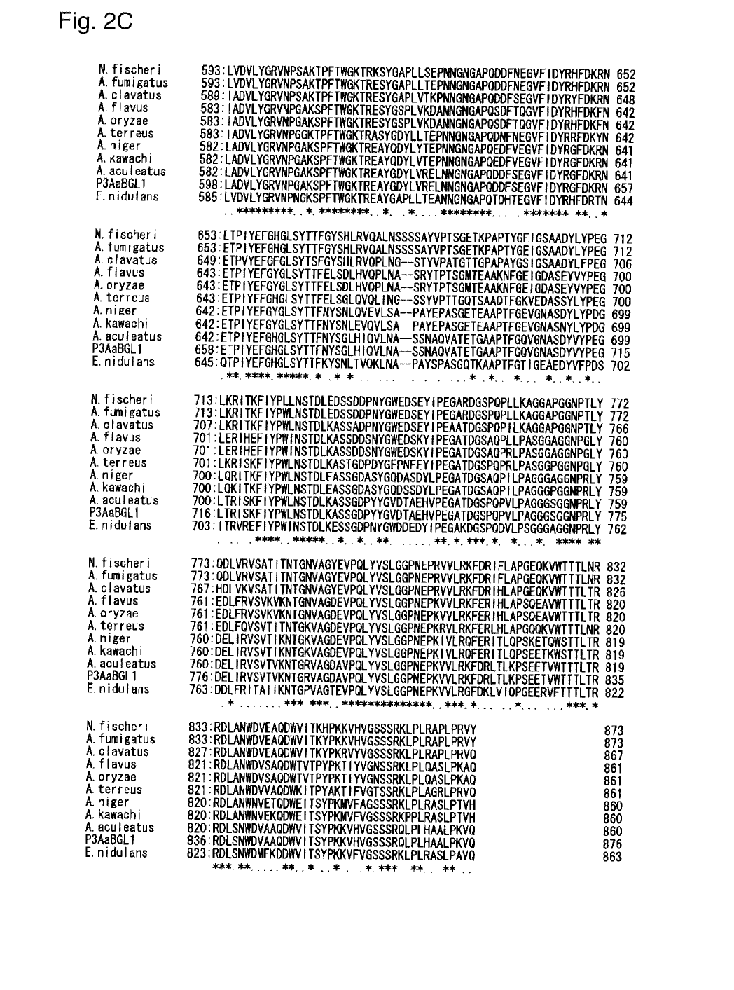

In the present specification and present invention, “amino acid corresponds to amino acid at position X of AaBGL1” in a certain β-glucosidase means an amino acid exists in a position corresponds to an amino acid at position X of AaBGL1, assuming that the amino acid sequence of AaBGL1 (SEQ ID NO: 1) and the amino acid sequence of the β-glucosidase are aligned in a manner to maximize their homology. FIG. 2 shows alignments of the amino acid sequence of AaBGL1 and the amino acid sequences of β-glucosidases derived from other filamentous fungi.

Specifically, as shown in FIG. 2, amino acids correspond to isoleucine at position 244 of AaBGL1 are valine at position 245 in Aspergillus oryzae or Aspergillus terreus, valine at position 244 in Aspergillus niger, and isoleucine at position 247 in Emericella nidulans.

Further, amino acids correspond to glycine at position 282 of AaBGL1 are threonine at position 283 in Aspergillus oryzae, serine at position 283 in Aspergillus terreus, alanine at position 282 in Aspergillus niger, and serine at position 285 in Emericella nidulans.

Further, amino acids correspond to asparagine at position 442 of AaBGL1 are asparagine at position 443 in Aspergillus oryzae, lysine at position 443 in Aspergillus terreus, asparagine at position 442 in Aspergillus niger, and glutamine at position 445 in Emericella nidulans.

Further, amino acids correspond to glycine at position 452 of AaBGL1 are glycine at position 453 in Aspergillus oryzae or Aspergillus terreus, glycine at position 452 in Aspergillus niger, and glycine at position 455 in Emericella nidulans.

Further, amino acids correspond to valine at position 503 of AaBGL1 are valine at position 504 in Aspergillus oryzae or Aspergillus terreus, valine at position 503 in Aspergillus niger, and valine at position 506 in Emericella nidulans.

Further, amino acids correspond to arginine at position 613 of AaBGL1 are lysine at position 614 in Aspergillus oryzae, threonine at position 614 in Aspergillus terreus, threonine at position 613 in Aspergillus niger, and threonine at position 616 in Emericella nidulans.

The wild-type β-glucosidase which does not have the mutations of the present may be a protein derived from a filamentous fungus and has a glucosidase activity, but is preferably BGL1 from the viewpoint of its high enzymatic activity. etc.

Further, the wild-type β-glucosidase may be derived from any filamentous fungus so long as it is derived from a filamentous fungus, but is preferably a β-glucosidase derived from a filamentous fungus of the genus Aspergillus such as a β-glucosidase derived from Aspergillus oryzae, or Aspergillus aculeatus. Among them, a β-glucosidase derived from Aspergillus aculeatus is preferred since it has high crystalline cellulose degradation ability and high yield of monosaccharide, and AaBGL1 is more preferred. According to the doctoral dissertation of Dr. Reiichiro Sakamoto (Research on cellulase system of Aspergillus aculeatus No. F-50, Osaka Prefecture University, 1984), the wild type AaBGL1 purified from Aspergillus aculeatus has a molecular weight of about 133 kDa, an optimal pH of 4.0, and a stable pH range of from 3 to 7 (25° C., 24 hours).

The modified-type β-glucosidase of the present invention may, as compared to the wild-type β-glucosidase derived from a filamentous fungus, specifically be one having at least one mutation among the above-described mutations 1 to 6, or one having, in addition to any one of the above-described mutations 1 to 6, other mutations or modifications so long as they do not impair the effects of mutations 1 to 6 and the glucosidase activity. As the modified-type β-glucosidase having such other mutations, etc., one having deletion, substitution or addition of at least one amino acid, preferably from one to tens amino acids, more preferably from one to dozen amino acids, further preferably from one to nine amino acids, even further preferably from one to few amino acids, and having at last one mutation selected from the group consisting of the above-described mutations 1 to 6 in the protein showing a glucosidase activity may, for example, be mentioned. For example, one having deletion of the N-terminal side or C-terminal side region of wild-type β-glucosidase which is comprised of from one to tens amino acids and does not affect glucosidase activity and having at least one mutation selected from the group consisting of the above-described mutations 1 to 6, may be used as the modified-type β-glucosidase.

The modified-type β-glucosidase of the present invention may be one having a homology to the amino acid sequence of SEQ ID NO: 1 of at least 60%, preferably at least 65%, more preferably at least 70%, further preferably at least 75%, even further preferably at least 80%, and having at last one mutation selected from the group consisting of the above-described mutations 1 to 6 in the protein showing a catalytic activity to hydrolyze a β-D-glucopyranoside bond.

### (Host)

The host of the transformant of the present invention is a yeast of the genus Schizosaccharomyces. The yeast of the genus Schizosaccharomyces to be used in the present invention may be a wild-type or a mutant-type in which a specific gene is deleted or inactivated depending on application. For deletion or inactivation of a specific gene, conventional methods can be used. Specifically, the Latour system (Nucleic Acids Res. (2006) 34: e11, and WO2007/063919) can be used to delete the gene. Further, the gene can be inactivated by mutating the gene at a certain position by mutant screening using mutagens (Koubo Bunshi Idengaku Jikken-Hou, 1996, Japan Scientific Societies Press), random mutations using PCR (polymerase chain reaction) (PCR Methods Appl., 1992, vol. 2, p. 28-33) and the like. As the yeast of the genus Schizosaccharomyces host in which a specific gene is deleted or inactivated, ones disclosed in WO2002/101038, WO2007/015470, etc. may, for example, be used.

Further, it is preferred to use a yeast of the genus Schizosaccharomyces host having a marker for selecting a transformant. For example, it is preferred to use a host which essentially requires a specific nutrient factor for growth due to deletion of a gene. When preparing a transformant by using a vector containing a target gene sequence, a transformant lacking the auxotrophy of the host can be obtained by using a vector carrying the deleted gene (auxotrophic complementation marker). It is possible to select the transformant by using the difference in auxotrophy between the host and the transformant.

For example, a yeast of the genus Schizosaccharomyces host which has been made auxotrophic for uracil by deletion or inactivation of orotidine 5′-phosphate decarboxylase gene (ura4 gene) is transformed with a vector containing ura4 gene (auxotrophic complementation marker), and transformants carrying the vector are obtained by selecting ones lacking uracil auxotrophy. The gene to be deleted to make an auxotrophic host is not limited to ura4 gene when it is used for selection of a transformant, and may, for example, be isopropyl malate dehydrogenase gene (leu1 gene).

As the yeast of the genus Schizosaccharomyces, Schizosaccharomyces pombe, Schizosaccharomyces japonicus, and Schizosaccharomyces octosporus may, for example, be mentioned. Among the above-mentioned yeasts of the genus Schizosaccharomyces, Schizosaccharomyces pombe is preferred in view of the availability of various useful mutant strains.

### (Expression Cassette)

The expression cassette is a combination of DNA essential for expressing a protein, and contains a structural gene sequence encoding the protein, and a promoter and a terminator capable of functioning in yeast of the genus Schizosaccharomyces.

The transformant of the present invention is prepared by introducing an expression cassette containing a structural gene sequence comprising a region encoding the above-described modified-type β-glucosidase (modified-type β-glucosidase structural gene sequence), and a promoter sequence and a terminator sequence for expressing the structural gene, into a yeast of the genus Schizosaccharomyces.

The expression cassette may additionally contain at least one of a 5′-untranslated region and a 3′-untranslated region. The expression cassette preferably contains all of the modified-type β-glucosidase structural gene sequence, the promoter, the terminator, the 5′-untranslated region and the 3′-untranslated region. Further, genes such as an auxotrophic complementation marker may be contained.

The nucleotide sequence of the region encoding the modified-type β-glucosidase in the modified-type β-glucosidase structural gene sequence is obtained by introducing a nucleotide substitution which reflects at least one amino acid substitution selected from the group consisting of mutations 1 to 6, and other modification as the case requires, into a structural gene of a wild-type β-glucosidase by using a genetic engineering method. Further, a gene encoding β-glucosidase derived from a filamentous fungus may be used as it is for a portion excluding the portion where the above-described modification is introduced. However, to increase expression in yeast of the genus Schizosaccharomyces, it is preferred to modify the above-described gene sequence by changing its codons to ones frequently used in a gene highly expressed in yeast of the genus Schizosaccharomyces.

The modified-type β-glucosidase structural gene sequence may contain, in addition to a region encoding the modified-type β-glucosidase, a region encoding other peptide or protein. In such a case, the transformant of the present invention produces a protein in which such other peptide is bound to the N-terminal or C-terminal of the modified-type β-glucosidase. As the peptide to be bound to the modified-type β-glucosidase, a signal such as a secretion signal, an organelle localization signal or the like, and a tag such as His-tag or FLAG-tag may, for example, be mentioned. The signal should be a signal capable of functioning in yeast of the genus Schizosaccharomyces.

The secretion signal is a peptide introduced at the N-terminal and having a function of secreting the expressed protein out of the host cell. As the secretion signal capable of functioning in yeast of the genus Schizosaccharomyces, P3 signal described in WO1996/23890 is particularly preferred.

The modified-type β-glucosidase structural gene sequence is preferably a sequence in which a region encoding a secretion signal is bound to the 5′ end side of the region encoding the modified-type β-glucosidase. By having a secretion signal at the N-terminal, it becomes possible to secrete the modified-type β-glucosidase produced in the transformant out of the cells of a yeast of the genus Schizosaccharomyces. The modified-type β-glucosidase secreted out of the cells can be easily collected and purified from the supernatant of a culture broth.

The promoter and the terminator may be ones capable of functioning in a yeast of the genus Schizosaccharomyces host and can direct expression of the modified-type β-glucosidase. As the promoter capable of functioning in yeast of the genus Schizosaccharomyces, a promoter intrinsic to the yeast of the genus Schizosaccharomyces (preferably one having a high transcriptional activity) or a promoter extrinsic to the yeast of the genus Schizosaccharomyces (such as a promoter derived from a virus) may be used. Further, two or more types of promoters may be contained in the vector.

As the promoter intrinsic to the yeast of the genus Schizosaccharomyces, a promoter of alcohol dehydrogenase gene, a promoter of nmt1 gene which relates to thiamine metabolism, a promoter of fructose 1,6-bisphosphatase gene which relates to glucose metabolism, a promoter of an invertase gene which relates to catabolite repression (WO99/23223) or a promoter of a heat shock protein gene (WO2007/26617) may, for example, be mentioned.

As the promoter extrinsic to the yeast of the genus Schizosaccharomyces, a promoter derived from an animal cell virus disclosed in JP-A-5-15380, JP-A-7-163373 or JP-A-10-234375 may, for example, be mentioned, and hCMV promoter or SV40 promoter is preferred.

As the terminator capable of functioning in yeast of the genus Schizosaccharomyces, a terminator intrinsic to the yeast of the genus Schizosaccharomyces, or a terminator extrinsic to the yeast of the genus Schizosaccharomyces, may be used. Further, two or more types of terminators may be contained in the vector.

As the terminator, the terminator derived from the human disclosed in JP-A-5-15380, JP-A-7-163373 or JP-A-10-234375 may, for example, be mentioned, and human lipocortin-I terminator is preferred.

### [Transformation Method]

The transformation method for a yeast of the genus Schizosaccharomyces of the present invention (hereinafter referred to as transformation method of the present invention) is characterized in that the yeast of the genus Schizosaccharomyces is transformed by using a vector containing the above-described expression cassette.

The yeast of the genus Schizosaccharomyces to be used as a host, modified-type β-glucosidase, filamentous fungus, and expression cassette containing the modified-type β-glucosidase structural gene sequence are as described above.

### (Vector)

The vector to be used for the transformation method of the present invention contains the above-described expression cassette. Further, a selection marker may preferably be contained in the vector. For example, a vector containing an auxotrophic complementation marker proper for the auxotrophy of the host is preferably used.

Further, the vector of the present invention preferably contains a secretion signal gene capable of functioning in yeast of the genus Schizosaccharomyces. The secretion signal gene is located at the 5′ end side of the structural gene sequence encoding β-glucosidase. A heterologous protein to which the secretion signal is attached at its N-terminal is expressed from a heterologous protein structural gene to which the secretion signal gene is bound at its 5′ end side. The secretion signal is removed from the protein in the endoplasmic reticulum and the Golgi apparatus, etc. of the host cell, and then, the heterologous protein detached from the secretion signal is secreted out of the host cell. The secretion signal gene (and the secretion signal) should be capable of functioning in yeast of the genus Schizosaccharomyces. As the secretion signal gene capable of functioning in yeast of the genus Schizosaccharomyces, the secretion signal genes described in WO1996/23890 may be used.

The vector to be used for the transformation method of the present invention is a vector having a circular DNA structure or a linear DNA structure. In the case of preparing a transformant in which the above-described expression cassette is maintained in the cells of yeast of the genus Schizosaccharomyces as an extrachromosomal gene, the vector is preferably a plasmid which contains a sequence required for replication in yeast of the genus Schizosaccharomyces, i.e. Autonomously Replicating Sequence (ARS).

On the other hand, in the case of preparing a transformant in which the above-described expression cassette is integrated into the chromosomes of yeast of the genus Schizosaccharomyces, the vector is preferably introduced into the host cells in the form of a linear DNA structure.

A transformant in which the expression cassette is integrated into the chromosomes of yeast of the genus Schizosaccharomyces is preferred from the viewpoint of subculture passage of the transformant. The integrated expression cassettes are unlikely to be lost, whereby the passage stability becomes quite high. That is, the transformant in which the expression cassette is integrated into its chromosomes can produce a heterologous protein with more stable productivity.

Here, the vector for preparing the transformant in which the expression cassette is integrated into the chromosomes of yeast of the genus Schizosaccharomyces will be described.

The integration of the expression cassette into the chromosomes is preferably carried out by homologous recombination since it is possible to integrate the expression cassette into any position of the chromosomes by homologous recombination.

When introducing the vector into the host cells in the form of a linear DNA structure, in the case of using a vector having a circular DNA structure like a usual plasmid DNA, it is preferred that the vector is cut open to a linear form by a restriction enzyme before introduction to yeast of the genus Schizosaccharomyces cells. In this case, the vector having a circular DNA structure is cut open at a position within the recombination region. The resulting vector has parts of the recombination regions exist at both ends and is integrated entirely into the target site in the chromosomes by homologous recombination.

The vector may be constructed by other methods without cutting a vector having a circular DNA structure, for example, by enzymatic amplification using PCR or a chemical synthesis, so long as a linear DNA structure having parts of the recombination region at both ends can be obtained.

To construct the vector of the present invention, a plasmid derived from E. coli such as pBR322, pBR325, pUC118, pUC119, pUC18, pUC19 or the like may suitably be used. A vector constructed by using a plasmid derived from E. coli usually has the replication origin region called “ori” which is necessary for replication in E. coli. Further, even in a case where a plasmid derived from E. coli like those mentioned above is not used, when E. coli is used for construction and amplification of the vector of the present invention, the “ori” is utilized to obtain a vector containing “ori”.

It is preferred that the replication origin region called “ori” required for replication in E. coli is removed from the vector for homologous recombination. Accordingly, it is possible to improve the integration efficiency at the time of integrating the above-described vector into a chromosome (refer to JP-A-2000-262284).

The method for constructing the vector in which the replication origin region is removed is not particularly limited, but is preferably the method disclosed in JP-A-2000-262284. That is, it is preferable to preliminarily construct a precursor vector carrying the replication origin region at a position to be cut within the recombination region so that the replication origin region will be cut-off from the vector at the time of preparing a linear DNA structure. Thus, a vector in which the replication origin region is removed is obtained easily.

Further, it may be a method wherein a precursor vector containing an expression cassette and a recombination region is constructed by using the vectors and their construction methods disclosed in JP-A-5-15380, JP-A-7-163373, WO96/23890, JP-A-10-234375, and then the replication origin region is removed from the precursor vector by using a usual genetic engineering method to obtain a vector to be used for homologous recombination.

The number of copies of the expression cassette in the vector may be only one, or two or more. The vector to be used for the transformation method of the present invention preferably contains from 1 to 8, particularly preferably from 2 to 4 copies of the expression cassette.

When the number of copies of the expression cassette in the vector is at least 2, it becomes easier to increase the number of copies of the expression cassette integrated into the chromosomes of yeast of the genus Schizosaccharomyces and expression of a heterologous protein. For example, when the number of copies of the expression cassette in the vector is at least 2, a plurality of the expression cassettes may be integrated sequentially in the same position of the chromosomes of yeast of the genus Schizosaccharomyces. Further, when the number of copies of the expression cassette in the vector is at most 8, reduction in the efficiency of vector integration via homologous recombination, which happens in a case where the vector size is too large, is likely to be suppressed. When the number of copies of the expression cassette is at most 4, the vector integration efficiency can be improved further.

When the number of the after-mentioned target site in the chromosomes is large, many copies of the expression cassette can be integrated even if the vector has one copy of the expression cassette. Further, two or more copies of the expression cassette may be integrated into the same position of the chromosomes of yeast of the genus Schizosaccharomyces.

Further, by setting the number of copies of the expression cassette in the vector to one, and the number of the after-mentioned target site in the chromosomes to one, the amount of modified-type β-glucosidase expressed in each transformant can be adjusted to a uniform level. For example, when evaluating the effect of the introduction of various mutations on the glucosidase activity, by adjusting the amount of modified-type β-glucosidase expressed in each transformant to a uniform level, and then comparing the glucosidase activity on a dry cell weight basis of each transformant (or on a supernatant weight basis in a case where the modified-type β-glucosidase is secreted out of the cells), the glucose activities of modified-type β-glucosidase produced in various transformants can be compared for evaluation.

The recombination region of the vector is a region having a nucleotide sequence which can induce homologous recombination with a target site in the chromosomes of yeast of the genus Schizosaccharomyces at which homologous recombination is to be achieved. Further, the target site is a site to become a target for integration of an expression cassette in the chromosomes of yeast of the genus Schizosaccharomyces. The target site can be designed freely by letting the recombination region of the vector have a nucleotide sequence which induces homologous recombination with the target site.

To induce homologous recombination, the recombination region is required to have a nucleotide sequence with a homology of at least 70% with the target site. Further, the nucleotide sequence homology between the recombination region and the target site is preferably at least 90%, more preferably at least 95%, in view of increasing the efficiency of homologous recombination. By using a vector containing such a recombination region, the expression cassette is integrated into the target site by homologous recombination.

The length of the recombination region is preferably from 20 to 2,000 bp. When the length of the recombination region is at least 20 bp, homologous recombination is likely to be induced. Further, when the length of the recombination region is at most 2,000 bp, reduction in the homologous recombination efficiency due to too large vector size is likely to be prevented. The length of the recombination region is preferably at least 100 bp, more preferably at least 200 bp. Further, the length of the recombination region is preferably at most 800 bp, more preferably at most 400 bp.

### (Target Site in Host)

When integrating only one copy of the expression cassette into the chromosomes of the host, the target site in all chromosomes of yeast of the genus Schizosaccharomyces for integration of the expression cassette is preferably a target site which located at a position where the integration does not lead to lost or inactivation of an essential gene. When the number of the target site is one, the number of copies of the expression cassette integrated into the chromosomes is fixed and then the comparison among various mutant strains becomes easier, whereby the selectivity of the modified-type β-glucosidase increases.

The above-mentioned essential gene is a gene whose lost or inactivation leads to inviability, i.e. a gene essential for growth of the transformant of the present invention.

When integrating a plurality of the expression cassettes into the chromosomes of the host, the target site for integration of the expression cassette is preferably a plurality of target sites in all chromosomes of yeast of the genus Schizosaccharomyces, which satisfies either one of conditions (1) two or more target sites exist in different chromosomes or (2) two or more target sites exists at a plurality of positions in the same chromosome separated by at least one essential gene, or both of (1) and (2). These two or more target sites have substantially the same nucleotide sequence. Here, “substantially the same nucleotide sequence” means that there is at least 90% nucleotide sequence homology between target sites. The nucleotide sequence homology between target sites is preferably at least 95%, more preferably at least 99%.

When the transformant loses the introduced expression cassettes which are separated by the essential gene, the essential gene is also lost, whereby the transformant cannot grow. Accordingly, during cultivation, the transformant which lost the expression cassettes may not grow along with the transformant retaining the expression cassettes, whereby the heterologous protein production efficiency is unlikely to be reduced. Because the growth rate of the transformant which lost expression cassettes has a higher growth rate in general, it is preferred to introduce the expression cassettes into target sites which satisfy the above condition (2).

As described above, since the target sites are dispersed in the chromosomes of yeast of the genus Schizosaccharomyces, the expression cassettes are integrated into the chromosomes in a dispersed manner, whereby the integrated expression cassettes are unlikely to be lost and their passage stability is quite high. Accordingly, it is possible to produce a heterologous protein stably with high productivity.

Further, by designing the target site so as to have substantially the same nucleotide sequence, even in a case where a plurality of target sites exists in different positions, it becomes possible to easily integrate the vector into the respective target sites by using a single type of vector.

In the transformation method of the present invention, when integrating a plurality of the expression cassettes, the number of target site positions where the vector is to be integrated is preferably at least 5. When the number of target site positions is at least 5, it is easier to increase the number of copies of the expression cassette integrated into the chromosomes, whereby the productivity of a heterologous protein increases further.

Further, the number of target site positions is preferably from 10 to 60. When the number of target site positions is at least 10, it is easier to further increase the number of copies of the expression cassette integrated into the chromosomes, whereby the productivity of a heterologous protein increases further. When the number of target site positions is at most 60, reduction in expression of a heterologous protein due to integration of too many copies of the expression cassette into the chromosomes is likely to be suppressed.

The target sites preferably have a nucleotide sequence which exists in a transposon gene Tf2, since it becomes possible to integrate the expression cassettes into the target sites dispersed at plural positions in the chromosomes of yeast of the genus Schizosaccharomyces at a time by using a single type of vector. Tf2 is a transposon gene which has a length (the number of nucleotide) of about 4,900 bp and exists in the three chromosomes of yeast of the genus Schizosaccharomyces at 13 positions in total, with a nucleotide sequence homology of 99.7% (refer to Nathan J. Bowen et al, Genome Res. 2003 13: p 1984-1997).

However, the target sites are not limited to the above-described ones. Other than the transposon gene Tf2, the target sites may, for example, be sites (such as genes) found at plural positions in the chromosomes, and having a length larger than the length of the recombination region and a substantially identical nucleotide sequence. Further, for example, after formation of the target site by newly introducing a plurality of genes (target sites) having a substantially identical nucleotide sequence and a length larger than the length of the recombination region into the chromosomes, the vector may be introduced into a plurality of target sites.

### (Transformation Method)

The yeast of the genus Schizosaccharomyces host is transformed by using the above-described vector. As the transformation method, any known transformation method for yeast of the genus Schizosaccharomyces may be used. Such a transformation method may, for example, be a conventional method like a lithium acetate method, electroporation method, spheroplast method, glass-beads method, or the like, and a method disclosed in JP-A-2005-198612. Further, a commercially available yeast transformation kit may be used.

After transformation, the resulting transformants are usually subjected to selection. The selection may, for example, be carried out as follows. Several transformants are selected as viable colonies in a broth via the above-mentioned auxotrophic marker screening method, the transformants are grown separately in a liquid broth, and transformants highly expressing a heterologous protein are selected by measuring the amount of a heterologous protein expressed in each transformant (or secreted to a supernatant in a case where the heterologous protein is secreted out of the cells). The number of vectors and copies of the expression cassette integrated into the chromosomes can be identified by analyzing the genomes of the selected transformants by pulse-field gel electrophoresis.

### (Cultivation Method)

The transformant of the present invention may be cultivated in the same manner as a natural yeast of the genus Schizosaccharomyces.

As the culture broth for cultivating the transformant of the present invention, a known culture broth for yeasts may be used so long as it contains carbon sources, nitrogen sources, inorganic salts and the like which yeast of the genus Schizosaccharomyces can use, and yeast of the genus Schizosaccharomyces can grow in it efficiently. The culture broth may be natural or synthetic.

As the carbon sources, saccharides such as glucose, fructose and sucrose may, for example, be mentioned.

As the nitrogen sources, inorganic acids or inorganic ammonium salts such as ammonia, ammonium chloride, and ammonium acetate, peptone and casamino acid may, for example, be mentioned.

As inorganic salts, magnesium phosphate, magnesium sulfate and sodium chloride may, for example, be mentioned.

Cultivation may be carried out by using a known cultivation method for yeasts such as a shaking cultivation, a stirring cultivation or the like.

The cultivation temperature is preferably from 23 to 37° C. Further, the cultivation time may be set appropriately.

Cultivation may be carried out batch-wise or continuously.

The transformant of the present invention is a yeast of the genus Schizosaccharomyces which can which can produce a modified-type β-glucosidase having higher thermostability or higher resistance to glucose inhibition than a wild-type β-glucosidase. The transformant of the present invention can produce the modified-type β-glucosidase, whereby it can utilize cellobiose.

### [Method for Producing β-Glucosidase]

The modified-type β-glucosidase can be recovered from the cells obtained by cultivating the transformant of the present invention. When the transformant is cultivated to isolate the modified-type β-glucosidase from culture broth or cells, a known protein isolation method may be used. For example, the cells are separated from the culture broth after cultivation, and then the separated cells are disrupted to obtain a cell lysate containing the modified-type β-glucosidase, followed by recovery of the modified-type β-glucosidase from the cell lysate by using a known protein isolation method such as salting-out, column chromatography purification or immunoprecipitation.

Further, in the case of using a transformant in which an expression cassette containing a structural gene sequence comprising a region encoding the modified-type β-glucosidase and a region encoding a secretion signal attached at the 5′ end of the region encoding the modified-type β-glucosidase is introduced, the modified-type β-glucosidase produced by the transformant is secreted out of the cells. Therefore, it is possible to recover the modified-type β-glucosidase from a culture supernatant by using a known protein isolation method. The transformant may be cultivated continuously to produce the modified-type β-glucosidase by repeating recovery of a culture supernatant containing the modified-type β-glucosidase from the culture broth by means of centrifugation or the like after a certain period of cultivation, and re-cultivation of the remained cells after supplementing them with a culture broth.

### [Cellulose Degradation Method]

In the cellulose degradation method of the present invention, the modified-type β-glucosidase recovered from the cells or the culture broth obtained by cultivating the above-described transformant of yeast of the genus Schizosaccharomyces is used.

The form of cellulose to be degraded is not particularly limited, and may be purified cellulose or cellulose contained in a biomass such as a wood, rice straw, rice husk and weed.

As the specific cellulose degradation method, a method wherein the transformant is cultivated to isolate or purify a modified-type β-glucosidase from the culture broth or the cells, and then the isolated or purified modified-type β-glucosidase is cultivated along with a biomass containing cellulose, an endoglucanase, and a cellobiohydrolase may, for example, be mentioned. In the case of decomposing cellulose by using the isolated and purified modified-type β-glucosidase produced by the transformant, the reaction conditions may be known reaction conditions for β-glucosidase. The suitable pH for the modified-type β-glucosidase in the degradation reaction solution is from 3.0 to 8.0, and the suitable reaction temperature is from 20 to 65° C.

The above-mentioned culture supernatant or cell lysate may be directly contacted with cellulose without isolating the modified-type β-glucosidase from the culture supernatant or cell lysate containing the modified-type β-glucosidase. For example, in the case of using a transformant in which an expression cassette containing a region encoding the modified-type β-glucosidase and a region encoding a secretion signal at the 5′ end of the region encoding the modified-type β-glucosidase is introduced, the transformant may be cultivated in a culture broth containing cellulose thereby to degrade the cellulose contained in the culture broth by the modified-type β-glucosidase secreted into the culture broth. As the specific cellulose degradation method, a method wherein the transformant is cultivated in the presence of the above-mentioned biomass, a method wherein the transformant is cultivated and then mixed with the above-mentioned biomass for degradation, etc. may, for example, be mentioned. In such a case, a general cultivation condition for yeast of the genus Schizosaccharomyces may be used as the cultivation condition. The suitable pH of the culture broth is from 3.0 to 8.0, and the suitable cultivation temperature is from 23 to 37° C.

### EXAMPLES

Now, the present invention will be described in further detail with reference to Examples and Comparative Examples. However, it should be understood that the present invention is by no means thereby restricted.

### Example 1

### Preparation of a Schizosaccharomyces pombe Transformant which can Produce AaBGL1

A gene sequence was designed based on the peptide sequence of AaBGL1 represented by SEQ ID NO: 1, by replacing the codons with codons highly expressed in Schizosaccharomyces pombe (refer to SEQ ID NO: 2. hereinafter referred to as AaBGL1 gene). The recognition sequences for KpnI and BspHI were added upstream of the initiation codon. The recognition sequences for XbaI and SacI were added downstream of the stop codon. A plasmid containing these sequences (synthesized by Geneart AG, Regensburg, Germany) was digested with restriction enzymes BspHI and XbaI.

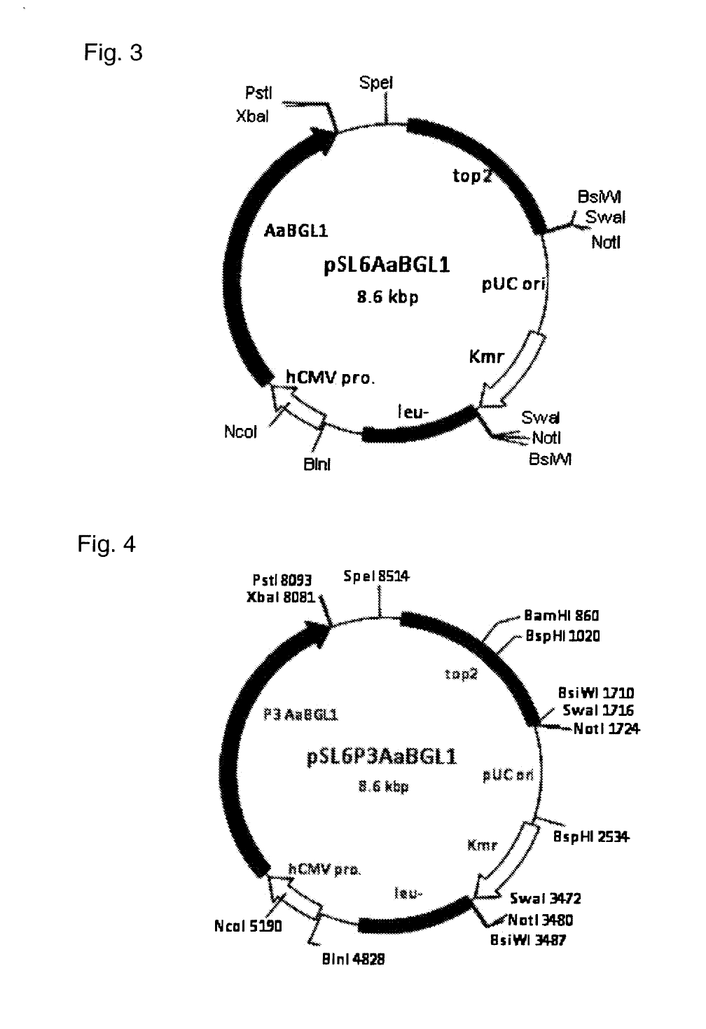

On the other hand, separately therefrom, pSL6lacZ was digested with restriction enzymes AarI and XbaI, and then treated with an alkaline phosphatase. Thereafter, gel electrophoresis was carried out on an agarose gel to isolate the digested fragment of vector pSL6 and the digested fragment of AaBGL1 gene from the agarose gel, and then these fragments were ligated to each other. The ligated product was introduced into E. coli DH5α (Takara Bio, Inc.) to obtain a transformant. From the obtained transformant, a vector was prepared to obtain expression vector pSL6AaBGL1 (FIG. 3, refer to SEQ ID NO: 3). The obtained expression vector was confirmed to be a desired vector by restriction enzyme mapping.

Further, to prepare P3AaBGL1 in which secretion signal P3 is bound at the N-terminal of AaBGL1 having a 19 amino acid deletion at its N-terminal (refer to SEQ ID NO: 4), a fragment of AaBGL1 gene was amplified by PCR method with In-fusion primers and pSL6AaBGL1 as template. On the other hand, pSL6P3lacZ was digested with restriction enzymes AflII and XbaI. The digested fragments and the PCR-amplified product of the AaBGL1 gene fragment were circularized by In-fusion method, and then introduced into E. coli DH5α (Takara Bio, Inc.) to obtain a transformant. From the obtained transformant, a vector was prepared to obtain expression vector pSL6P3AaBGL1 (FIG. 4, refer to SEQ ID NO: 5). The obtained expression vector was confirmed to be a desired vector by restriction enzyme mapping and partial nucleotide sequencing.

As the host cell, a leucine-auxotrophic strain of Schizosaccharomyces pombe (genotype: h-, leu1-32, provided from professor Yuichi lino, Molecular Genetics Research Laboratory, Graduate School of Science, The University of Tokyo) (ATCC38399) was cultivated in YES medium (0.5% of yeast extract, 3% of glucose and 0.1 mg/ml of SP supplements) until 0.6×10⁷ cells/ml. The cells were collected and washed, and then suspended by 0.1M lithium acetate solution (pH 5.0) to 1.0×10⁸ cells/ml. Thereafter, to 100 μl of the suspension, 1 μg of the above-mentioned vector pSL6P3AaBGL1 digested by restriction enzyme NotI was added, and then 290 μl of a 50% (w/v) polyethylene glycol (PEG4000) aqueous solution was added thereto, followed by stirring to cultivate them for 60 minutes at 30° C., 5 minutes at 42° C., and 10 minutes at room temperature, in this order. PEG4000 was removed by centrifugation and then the cells were washed to suspend them in 150 μl of sterile water. The suspension was applied on minimal-agarose medium.

Three days after cultivation, a transformant (AaBGL1 expression strain) was obtained. Thus obtained transformant was designated as ASP3326 strain.

### Preparation of Error-Prone PCR Fragment

By using pSL6AaBGL1 as template, a fragment of P3AaBGL1 gene was amplified by error-prone PCR method with the primers of SEQ ID NO: 6 and SEQ ID NO: 7. The error-prone PCR was carried out by using Diversify PCR Random Mutagenesis Kit (manufactured by Clontech Lab., Inc.). On the other hand, separately therefrom, by using pSL6P3AaBGL1 as a template, a fragment of leu1⁺ promoter was amplified by PCR method with the primers of SEQ ID NO: 8 and SEQ ID NO: 9. Further, separately therefrom, by using pSL6P3AaBGL1 as a template, a fragment of terminator top2 was amplified by PCR method with the primers of SEQ ID NO: 10 and SEQ ID NO: 11. For the PCR method, PrimeSTAR Max DNA polymerase (manufactured by Takara Bio Inc.) was used.

To a reaction mixture comprised of these three types of fragments, restriction enzyme DpnI was added to digest the templates, and then the fragments were purified by using SUPREC-PCR (manufactured by Takara Bio Inc.) for primer substitution. By using 50 ng of the purified three types of fragments, a mutant gene-integrated fragment comprising leu1⁺ promoter, mutant-type P3AaBGL1, and terminator top2 (leu1⁺ promoter-mutant-type P3AaBGL1-terminator top2) was amplified by PCR method with the primers of SEQ ID NO: 12 and SEQ ID NO: 13. Thereafter, a column purification was carried out by using SUPREC-PCR.

### Preparation of Transformant

As the host cell, a leucine-auxotrophic strain of Schizosaccharomyces pombe (genotype: h-, leu1-32, provided from professor Yuichi lino, Molecular Genetics Research Laboratory, Graduate School of Science, The University of Tokyo) (ATCC38399) was cultivated in YES medium (0.5% of yeast extract, 3% of glucose and 0.1 mg/ml of SP supplements) until 0.6×10⁷ cells/ml. The cells were collected and washed, and then suspended by 0.1M lithium acetate solution (pH 5.0) to 1.0×10⁸ cells/ml. Thereafter, to 100 μl of the suspension, 1 μg of the leu1⁺ promoter-P3AaBGL1-terminator top2 fragment was added, and then 290 μl of a 50% (w/v) polyethylene glycol (PEG4000) aqueous solution was added thereto, followed by stirring to cultivate them for 60 minutes at 30° C., 5 minutes at 42° C., and 10 minutes at room temperature, in this order. PEG4000 was removed by centrifugation and then the cells were washed to suspend them in 150 μl of sterile water. The suspension was applied on minimal-agarose medium. The composition of the minimal-agarose medium is shown in Tables 1 to 6.

By limiting the carbon sources of the minimal-agarose medium to 0.1% glucose and 2% cellobiose, it is possible to select an active type P3AaBGL1 mutant strain based on the formation of large colonies, since only the active type P3AaBGL1 mutant strain can utilize cellobiose as a carbon source.

Five days after cultivation, a transformant (P3AaBGL1 mutant strain) was obtained. The obtained transformant was designated as P3AaBGL1 mutant strain library.

TABLE 1
10xMA composition

| | | |
|---|---|---|
| Potassium hydrogen phthalate | 3 | g |
| Na₂HPO₄ | 2.2 | g |
| NH₄Cl | 5 | g |
| Water | Up to 1000 | ml |

TABLE 2
Salt stock solution composition

| | | |
|---|---|---|
| MgCl₂6H₂O | 53.3 | g |
| CaCl₂2H₂O | 0.735 | g |
| KCl | 50 | g |
| Na₂SO₄ | 2 | g |
| Water | Up to 1000 | ml |

TABLE 3
Mineral stock solution composition

| | | |
|---|---|---|
| H₃BO₃ | 500 | mg |
| MnSO₄5H₂O | 530 | mg |
| ZnSO₄7H₂O | 400 | mg |
| FeCl₃6H₂O | 200 | mg |
| Na₂MoO₄ 2H₂O | 200 | mg |
| KI | 100 | mg |
| CuSO₄5H₂O | 40 | mg |
| Potassium dihydrogen citrate | 1000 | mg |
| Water | Up to 1000 | ml |

TABLE 4
Vitamin stock solution composition

| | | |
|---|---|---|
| Pantothenic acid Ca | 0.01 | g |
| Nicotinic acid | 0.10 | g |
| Inositol | 0.10 | g |
| Water | Up to 10 | ml |

TABLE 5
Biotin stock solution composition

| | | |
|---|---|---|
| Biotin | 0.001 | g |
| Water | Up to 10 | ml |

TABLE 6
Minimal-agarose medium composition

| | | |
|---|---|---|
| 10xMA | 100 | ml |
| Salt stock solution | 20 | ml |
| Mineral stock solution | 1 | ml |
| Vitamin stock solution | 1 | ml |
| Biotin stock solution | 1 | ml |
| Glucose* | 20 | g |
| Agarose | 30 | g |
| Water | 880 | ml |

### Studies on Carbon Sources for Minimal-Agarose Medium

By limiting the carbon sources (indicated by * in Table 6) of the minimal-agarose medium to 0.1% glucose and 2% cellobiose, only an active type P3AaBGL1 mutant strain (transformant which produces an AaBGL1 mutant having a glucosidase activity) can utilize cellobiose as a carbon source. Accordingly, as described below, it becomes possible to select an active P3AaBGL1 mutant strain based on the formation of large colonies, by cultivating transformants in the minimal-agarose medium.

The colony formation and the BGL activity of a P3AaBGL1 mutant strain were compared with the case where 2% glucose was used as a carbon source. The BGL activity of a P3AaBGL1 mutant strain formed by using a minimal-agarose medium having glucose as a carbon source was measured in accordance with the following activity measurement method, and a result, the percentage of mutant strains having a BGL activity were found to be 26.5%, based on the total colonies. In comparison to this result, 70.5% of P3AaBGL1 mutant strains, based on the total P3AaBGL1 mutant strains formed in the minimal-agarose medium having 0.1% glucose and 2% cellobiose as carbon sources, were found to form small colonies. The BGL1 activities of the remaining P3AaBGL1 mutant strains which formed relatively large colonies were measured in accordance with the following activity measurement method, and as a result, the percentage of AaBGL1 mutant strains having a BGL activity was found to be 83.9%. That is, by selectively recovering P3AaBGL1 mutant strains which formed large colonies in the minimal-agarose medium having 0.1% glucose and 2% cellobiose as carbon sources, it was possible to exclude P3AaBGL1 mutant strains which had lost a BGL activity. Further, in the case where the carbon source was only 2% cellobiose, the recovery efficiency of transformants were significantly decreased, whereby recovery of large number of mutant strains was difficult. This is presumably because the transformants cannot utilize cellobiose due to lack of carbon sources required for expressing mutant-type P3AaBGL1 retaining the activity.

### Screening from P3AaBGL1 Mutant Strain Library

A mutant strain which formed a large colony was selected from P3AaBGL1 mutant strain library. Then, thus selected strain and P3ASP3326 strain as the original strain were cultivated in 150 μl/well (96-well plate) of YES medium at 30° C. for two days. 50 μl of each culture supernatant was diluted by 150 μl of 50 mM acetate buffer (pH 5.0) to prepare a diluted enzyme sample. Then, the enzymatic activity of the sample was measured in accordance with the following method.

### (Activity Measurement Method)

To 10 μl of 20 mM p-nitrophenyl-β-D-glucoside (hereinafter abbreviated as pNPG), 10 μl of 1M sodium acetate buffer solution (pH 5.0) and 130 μl of water were added, and then 50 μl of the diluted enzyme sample was introduced for reacting them at 37° C. for 10 minutes. 100 μl of the reaction mixture was mixed with 100 μl of 2% sodium carbonate solution to terminate reaction, and then the amount of free p-nitrophenol was colorimetrically measured at a wavelength of 450 nm.

The amount of enzyme that produces 1 μmol of p-nitrophenol per minute was defined as 1 U.

### (Glucose Inhibition Measurement)

To 10 μl of 20 mM pNPG, 10 μl of 1M sodium acetate buffer solution (pH 5.0) and from 4 to 10 μl of 1M glucose (final concentration of from 20 to 50 mM) were added, followed by addition of water up to the total volume of 150 μl. Thereafter, 50 μl of the diluted enzyme sample was introduced for reacting them at 37° C. for 10 minutes. As a control, a glucose-free reaction mixture was used, and then the reaction was carried out at 37° C. for 10 minutes, in the same manner. 100 μl of each reaction mixture was mixed with 100 μl of 2% sodium carbonate solution to terminate reaction, and then the amount of liberated p-nitrophenol was colorimetrically measured at a wavelength of 450 nm.

The ratio (%) of the colorimetric value of each reaction mixture to the colorimetric value of the control sample (glucose-free sample) was defined as the residual activity after glucose inhibition. The residual activities of each of the mutants were compared with the results obtained by using ASP3326 strain, thereby to select a mutant strain showed changes in resistance to glucose inhibition.

### (Thermostability Measurement)

To 10 μl of 20 mM pNPG, 10 μl of 1M sodium acetate buffer solution (pH 5.0) and 130 μl of water were added, and then 50 μl of a diluted enzyme sample preliminarily heat-treated at from 65 to 70° C. for 10 minutes was introduced for reacting them at 37° C. for 10 minutes. As a control, a diluted enzyme sample which was not subjected to the heat treatment was used, and then the reaction was carried out at 37° C. for 10 minutes, in the same manner. 100 μl of each reaction mixture was mixed with 100 μl of 2% sodium carbonate solution to terminate reaction, and then the amount of liberated p-nitrophenol was colorimetrically measured at a wavelength of 450 nm.

The ratio (%) of the colorimetric value of each reaction mixture to the colorimetric value of the control sample (untreated sample) was defined as the residual activity after heat treatment. The residual activities of each of the mutants were compared with the results obtained by using ASP3326 strain, thereby to select a mutant strain showed improved thermostability.

### Cultivation of Recovered Mutant Strain

The P3AaBGL1 mutant strain obtained as above was cultivated in YES medium at 30° C. for one day. 100 μl of the culture broth was inoculated on 5 ml of YPD medium (1% of yeast extract, 2% of peptone, and 2% of glucose), followed by cultivation at 30° C. for two days. Thereafter, the culture broth was centrifuged to obtain a culture supernatant.

### Evaluation of Recovered Mutant Strain

By using the above-obtained culture supernatant of the P3AaBGL1 mutant strain, a diluted enzyme sample was prepared, and then the enzymatic activity of the sample was measured in accordance with the above-described activity measurement method. Further, the glucose inhibition at a final glucose concentration of from 0.1 to 200 mM and the thermostability at a warming temperature of from 50 to 75° C. were measured for evaluation.

### Recovery of Modified-Type P3AaBGL1 Gene

From the P3AaBGL1 mutant strains obtained as above, a P3AaBGL1 mutant strain showed changes in the resistance to glucose inhibition and the thermostability of glucosidase, as compared to those of ASP3326 strain, was selected. 100 μl of the culture broth of the selected P3AaBGL1 mutant strain was centrifuged to collect cells. The collected cells were treated with Zymolyase, and thus treated cells were used as templates for amplifying a gene-integrated fragment by PCR method using the primers of SEQ ID NO: 14 and SEQ ID NO: 15, and PrimeSTAR Max DNA polymerase. Further, by using thus obtained fragment as a template, a fragment of P3AaBGL1 mutant gene was amplified by PCR method with In-fusion primers (the primers of SEQ ID NO: 16 and SEQ ID NO: 17). On the other hand, pSL6P3lacZ was digested with restriction enzymes AflII and XbaI. The digested fragments and the PCR-amplified product of the P3AaBGL1 mutant gene fragment were circularized by In-fusion method, and then introduced into E. coli DH5α to obtain a transformant. From the obtained transformant, a vector was prepared to obtain expression vector pSL6P3AaBGL1 mutXX (XX indicates strain name).

The nucleotide sequences of each P3AaBGL1 gene in pSL6P3AaBGL1 mutXX vector and the P3AaBGL1 mutant gene fragment were confirmed to identify the mutation site.

The above-described operation was repeated independently four times, and as a result, seven P3AaBGL1 mutant strains having P3AaBGL1 in which mutations affecting the resistance to glucose inhibition and the thermostability of the glucosidase expressed from the modified-type P3AaBGL1 gene were introduced, were obtained. With regard to modified-type P3AaBGL1 and P3AaBGL1 produced by the respective P3AaBGL1 mutant strains, the types of mutation introduced into P3AaBGL1, the glucosidase activity, the residual glucosidase activity in a culture broth containing glucose at a concentration of 20 mM or 50 mM (a value relative to the value obtained when the glucose concentration was 0 mM), and the residual glucosidase activity after warming a culture broth for 10 minutes at a warming temperature of 65° C. or 67° C. (a value relative to the value obtained when the culture broth was not subjected to preliminary heat treatment) were measured, and the results are shown in Table 7. In the Table, “Activity” indicates glucosidase activity and “Silent” indicates the number of silent mutation found in each mutant strain. Further, “w. t.” indicates the results of ASP3326 strain. These results suggest that Q142 (glutamine at position 142, corresponding to glutamine at position 126 of AaBGL1), Q156 (glutamine at position 156, corresponding to glutamine at position 140 of AaBGL1), I260 (isoleucine at position 260, corresponding to isoleucine at position 244 of AaBGL1), G298 (glycine at position 298, corresponding to glycine at position 282 of AaBGL1), N458 (asparagine at position 458, corresponding to asparagine at position 442 of AaBGL1), G468 (glycine at position 468, corresponding to glycine at position 452 of AaBGL1), V519 (valine at position 519, corresponding to valine at position 503 of AaBGL1), and R629 (arginine at position 629, corresponding to arginine at position 613 of AaBGL1) of P3AaBGL1 are involved in the resistance to glucose inhibition or the thermostability.

TABLE 7

| | | Residual activity [%] | | | | | | | | | | | | | | | | |
|---|---|---|---|---|---|---|---|---|---|---|---|---|---|---|---|---|---|---|
| | Activity | Glc | Glc | Temp | Temp | Mutation site | | | | | | | | | | | | |
| | [mU/ml] | 20 mM | 50 mM | 65° C. | 67° C. | I19 | Q142 | Q156 | I260 | G298 | N458 | G468 | V519 | R629 | Q681 | T693 | D744 | Silent |
| w.t. | 680 | 40 | 22 | 61 | 44 | | | | | | | | | | | | | |
| 6H02 | 446 | 22 | 12 | 46 | 33 | | | | | S | | | | | H | | N | 1 |
| 6H05 | 525 | 41 | 23 | 73 | 59 | | | | | | | | A | | | | | 4 |
| 9H03 | 647 | 65 | 43 | 38 | 23 | | | L | F | F | | | | | | | | 4 |
| 9H08 | 358 | 45 | 26 | 72 | 65 | | | | | | | | A | L | | | | 0 |
| 11F10 | 322 | 84 | 72 | 7 | 2 | | | L | F | F | | S | | | | | | 3 |
| 18A08 | 337 | 44 | 26 | 82 | 75 | | | | | | S | | A | L | | A | | 1 |
| 18A12 | 445 | 44 | 26 | 82 | 77 | N | L | | | | | | A | L | | | | 3 |
| 22A12 | 501 | 43 | 25 | 83 | 75 | | L | | | | S | | A | L | | | | 1 |

### Example 2

### Preparation of G298X Site-Saturation Mutagenesis Fragment

Specific amino acid substitution of G298 was carried out with degenerate primers. By using pSL6P3AaBGL1 as a template, a leu1⁺-G298X fragment was amplified by PCR method with the primers of SEQ ID NO: 8 and SEQ ID NO: 18. On the other hand, a G298X-top2 fragment was amplified by PCR method with the primers of SEQ ID NO: 19 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-G298X-top2 fragment was amplified by PCR method with the primers of SEQ ID NO: 12 and SEQ ID NO: 13. Thereafter, a column purification was carried out by using SUPREC-PCR.

### Recovery of Mutant Strain Obtained by Site-Saturation Mutagenesis

In the same manner as in Example 1, except that the leu1⁺-G298X-top2 fragment was used as a mutant gene-integrated fragment, mutant strains were obtained, and then the glucose inhibition and the thermostability at a warming temperature of from 50 to 75° C. of the culture supernatants of the respective mutant strains were measured. Thereafter, a mutant strain showed changes in any of the activities as compared to ASP3326 strain was selected, and then the nucleotide sequence of P3AaBGL1 gene integrated into the chromosomes of the selected mutant strain was confirmed, thereby to identify an amino acid at position 298. As a result, in a P3AaBGL1 mutant strain showed changes in the resistance to glucose inhibition or the thermostability as compared to ASP3326 strain, the amino acid at position 298 was identified as serine, phenylalanine or proline.

### Example 3

### Preparation of V519X Site-Saturation Mutagenesis Fragment

Specific amino acid substitution of V519 was carried out with degenerate primers. By using pSL6P3AaBGL1 as a template, a leu1⁺-V519X fragment was amplified by PCR method with the primers of SEQ ID NO: 8 and SEQ ID NO: 20. On the other hand, a V519X-top2 fragment was amplified by PCR method with the primers of SEQ ID NO: 21 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-V519X-top2 fragment was amplified by PCR method with the primers of SEQ ID NO: 12 and SEQ ID NO: 13. Thereafter, a column purification was carried out by using SUPREC-PCR.

### Recovery of Mutant Strain Obtained by Site-Saturation Mutagenesis

In the same manner as in Example 1, except that the leu1⁺-V519X-top2 fragment was used as a mutant gene-integrated fragment, mutant strains were obtained, and then the glucose inhibition and the thermostability at a warming temperature of from 50 to 75° C. of the culture supernatants of the respective mutant strains were measured. Thereafter, a mutant strain showed changes in any of the activities as compared to ASP3326 strain was selected, and then the nucleotide sequence of P3AaBGL1 gene integrated into the chromosomes of the selected mutant strain was confirmed, thereby to identify an amino acid at position 519. As a result, in a P3AaBGL1 mutant strain showed changes in the resistance to glucose inhibition or the thermostability as compared to ASP3326 strain, the amino acid at position 519 was identified as alanine, glycine or cysteine.

### Example 4

### Preparation of Q142X Site-Saturation Mutagenesis Fragment

Specific amino acid substitution of Q142 was carried out with degenerate primers. By using pSL6P3AaBGL1 as a template, a leu1⁺-Q142X fragment was amplified by PCR method with the primers of SEQ ID NO: 8 and SEQ ID NO: 22. On the other hand, a Q142X-top2 fragment was amplified by PCR method with the primers of SEQ ID NO: 23 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-Q142X-top2 fragment was amplified by PCR method with the primers of SEQ ID NO: 12 and SEQ ID NO: 13. Thereafter, a column purification was carried out by using SUPREC-PCR.

### Recovery of Mutant Strain Obtained by Site-Saturation Mutagenesis

In the same manner as in Example 1, except that the leu1⁺-Q142X-top2 fragment was used as a mutant gene-integrated fragment, mutant strains were obtained, and then the glucose inhibition and the thermostability at a warming temperature of from 50 to 75° C. of the culture supernatants of the respective mutant strains were measured. Thereafter, a mutant strain showed changes in any of the activities as compared to ASP3326 strain was selected, and then the nucleotide sequence of P3AaBGL1 gene integrated into the chromosomes of the selected mutant strain was confirmed, thereby to identify an amino acid at position 142. As a result, in a P3AaBGL1 mutant strain showed changes in the resistance to glucose inhibition or the thermostability as compared to ASP3326 strain, the amino acid at position 412 was identified as asparagine or arginine.

### Example 5

### Preparation of G468X Site-Saturation Mutagenesis Fragment

Specific amino acid substitution of G468 was carried out with degenerate primers. By using pSL6P3AaBGL1 as a template, a leu1⁺-G468X fragment was amplified by PCR method with the primers of SEQ ID NO: 8 and SEQ ID NO: 24. On the other hand, a G468X-top2 fragment was amplified by PCR method with the primers of SEQ ID NO: 25 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-G468X-top2 fragment was amplified by PCR method with the primers of SEQ ID NO: 12 and SEQ ID NO: 13. Thereafter, a column purification was carried out by using SUPREC-PCR.

### Recovery of Mutant Strain Obtained by Site-Saturation Mutagenesis

In the same manner as in Example 1, except that the leu1⁺-G468X-top2 fragment was used as a mutant gene-integrated fragment, mutant strains were obtained, and then the glucose inhibition and the thermostability at a warming temperature of from 50 to 75° C. of the culture supernatants of the respective mutant strains were measured. Thereafter, a mutant strain showed changes in any of the activities as compared to ASP3326 strain was selected, and then the nucleotide sequence of P3AaBGL1 gene integrated into the chromosomes of the selected mutant strain was confirmed, thereby to identify an amino acid at position 468. As a result, in a P3AaBGL1 mutant strain showed changes in the resistance to glucose inhibition or the thermostability as compared to ASP3326 strain, the amino acid at position 468 was identified as serine.

The types of mutation introduced into P3AaBGL1, the glucosidase activity, the residual glucosidase activity in a culture broth containing glucose at a concentration of 20 mM or 50 mM (a relative value), and the residual glucosidase activity after warming a culture broth for 10 minutes at a warming temperature of 65° C. or 67° C. (a relative value) of the respective mutant strains obtained in Examples 2 to 5 are shown in Table 8.

TABLE 8

| | | Residual activity [%] | | | | | | | | |
|---|---|---|---|---|---|---|---|---|---|---|
| | Activity | Glc | Glc | Temp | Temp | Mutation site | | | | |
| | [mU/ml] | 20 mM | 50 mM | 65° C. | 67° C. | Q142 | G298 | G468 | V519 | Silent |
| w.t. | 680 | 40 | 22 | 61 | 44 | | | | | |
| 7G06 | 362 | 55 | 35 | 39 | 27 | | F | | | 0 |
| 7G09 | 518 | 22 | 11 | 44 | 28 | | S | | | 0 |
| 8A08 | 312 | 63 | 41 | 5 | 6 | | P | | | 0 |
| 7F01 | 209 | 48 | 26 | 67 | 57 | | | | G | 0 |
| 8F08 | 513 | 43 | 23 | 61 | 47 | | | | C | 0 |
| 8F11 | 544 | 42 | 24 | 70 | 58 | | | | A | 0 |
| 32A02 | 582 | 38 | 21 | 70 | 58 | N | | | | 0 |
| 32A03 | 575 | 38 | 20 | 57 | 51 | R | | | | 0 |
| 31C06 | 267 | 75 | 54 | 12 | 2 | | | S | | 0 |

As a result, G298F (substitution of glycine at position 298 to phenylalanine) was found to increase the resistance to glucose inhibition and decrease the thermostability of P3AaBGL1. Further, V519A (substitution of valine at position 519 to alanine) was found to significantly increase the thermostability, while the resistance to glucose inhibition of P3AaBGL1 was not affected thereby. Further, G468S (substitution of glycine at position 468 to serine) was found to increase the resistance to glucose inhibition and decrease the thermostability of P3AaBGL1. Further, it was suggested that the thermostability of P3AaBGL1 can be improved by Q142N (substitution of glutamine at position 142 to asparagine).

### Example 6

### Recombination of Mutant Genes by StEP Method

By using two or several types of pSL6P3AaBGL1 mutXX vectors (total 1 ng) as templates, fragments of P3AaBGL1 gene were amplified by a StEP method of Zhao et al. (Huimin Zhao, METHODS IN ENZYMOLOGY, Vol. 388, pp. 42-49, 2004) with the primers of SEQ ID NO: 6 and SEQ ID NO: 7, PrimeSTAR HS DNA Polymerase (Takara Bio Inc.) and the following PCR program: 30 cycles of 10 seconds at 98° C., 5 seconds at 55° C., and 20 seconds at 72° C. (2.6 kb), followed by 5 cycles of 10 seconds at 98° C., 5 seconds at 55° C., and 3 minutes at 72° C. (2.6 kb). On the other hand, separately therefrom, by using pSL6P3AaBGL1 as a template, a fragment of leu1⁺ promoter was amplified by PCR method with the primers of SEQ ID NO: 8 and SEQ ID NO: 9. Further, separately therefrom, by using pSL6P3AaBGL1 as a template, a fragment of terminator top2 was amplified by PCR method with the primers of SEQ ID NO: 10 and SEQ ID NO: 11. For the PCR method, PrimeSTAR Max DNA polymerase (manufactured by Takara Bio Inc.) was used.

To a reaction mixture comprised of these three types of fragments, restriction enzyme DpnI was added to digest the templates, and then the fragments were purified by using SUPREC-PCR (manufactured by Takara Bio Inc.) for primer substitution. By using 50 ng of the purified three types of fragments, a leu1⁺ promoter-P3AaBGL1-terminator top2 fragment was amplified with the primers of SEQ ID NO: 12 and SEQ ID NO: 13. Thereafter, a column purification was carried out by using SUPREC-PCR.

### Recovery of StEP Mutation-Recombination Strain

In the same manner as in Example 1, except that the above-prepared fragment was used as a mutant gene-integrated fragment, mutant strains were obtained, and then the glucose inhibition and the thermostability at a warming temperature of from 50 to 75° C. of the culture supernatants of the respective mutant strains were measured. Thereafter, a mutant strain showed changes in any of the activities as compared to ASP3326 strain was selected, and then the nucleotide sequence of P3AaBGL1 gene integrated into the chromosomes of the selected mutant strain was confirmed.

The types of mutation introduced into P3AaBGL1, the glucosidase activity, the residual glucosidase activity in a culture broth containing glucose at a concentration of 20 mM or 50 mM, and the residual glucosidase activity after warming a culture broth for 10 minutes at a warming temperature of 65° C. or 67° C. of the respective mutant strains obtained as above are shown in Table 9.

TABLE 9

| | | Residual activity [%] | | | | | | | | | | | | |
|---|---|---|---|---|---|---|---|---|---|---|---|---|---|---|
| | Activity | Glc | Glc | Temp | Temp | Mutation site | | | | | | | | |
| | [mU/ml] | 20 mM | 50 mM | 65° C. | 67° C. | Q142 | Q156 | I260 | G298 | N458 | V519 | R629 | T693 | Silent |
| w.t. | 680 | 40 | 22 | 61 | 44 | | | | | | | | | |
| 13C02 | 357 | 67 | 46 | 63 | 48 | | L | F | F | | A | L | | 0 |
| 22A12 | 501 | 43 | 25 | 83 | 75 | L | | | | S | A | L | | 1 |
| 24A01 | 460 | 43 | 25 | 84 | 74 | L | | | | S | A | L | A | 0 |
| 24A12 | 316 | 66 | 45 | 78 | 67 | | L | F | F | S | A | L | A | 0 |
| 27A10 | 341 | 67 | 45 | 76 | 68 | | L | F | F | S | A | L | | 0 |

### Example 7

### Preparation of Site-Saturation Mutagenesis Fragment

By using pSL6P3AaBGL1mut27A10 vector as a template, specific amino acid substitutions of Q142N and G468S were carried out with degenerate primers.

When carrying out specific amino acid substitution of Q142N, specifically, a leu1⁺-Q142X fragment was amplified by PCR method with the primers of SEQ ID NO: 8 and SEQ ID NO: 22. On the other hand, a Q142N-top2 fragment was amplified by PCR method with the primers of SEQ ID NO: 23 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-Q142N-top2 fragment was amplified by PCR method with the primers of SEQ ID NO: 12 and SEQ ID NO: 13. Thereafter, a column purification was carried out by using SUPREC-PCR.

Further, when carrying out specific amino acid substitution of G468S, a leu1⁺-Q142X fragment was amplified by PCR method with the primers of SEQ ID NO: 7 and SEQ ID NO: 24. On the other hand, a G4685-top2 fragment was amplified by PCR method with the primers of SEQ ID NO: 25 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-G468S-top2 fragment was amplified by PCR method with the primers of SEQ ID NO: 12 and SEQ ID NO: 13. Thereafter, a column purification was carried out by using SUPREC-PCR.

### Recovery of Mutant Strain Obtained by Site-Saturation Mutagenesis

In the same manner as in Example 1, except that each of the above-prepared fragments was used as a mutant gene-integrated fragment, mutant strains were obtained, and then the glucose inhibition and the thermostability at a warming temperature of from 50 to 75° C. of the culture supernatants of the respective mutant strains were measured.

The types of mutation introduced into P3AaBGL1, the glucosidase activity, the residual glucosidase activity in a culture broth containing glucose at a concentration of 20 mM or 50 mM, and the residual glucosidase activity after warming a culture broth for 10 minutes at a warming temperature of 65° C. or 67° C. of the respective mutant strains obtained as above are shown in Table 10.

TABLE 10

| | | Residual activity [%] | | | | | | | | | | | | |
|---|---|---|---|---|---|---|---|---|---|---|---|---|---|---|
| | Activity | Glc | Glc | Temp | Temp | Mutation site | | | | | | | | |
| | [mU/ml] | 20 mM | 50 mM | 65° C. | 67° C. | Q142 | Q156 | I260 | G298 | N458 | G468 | V519 | R629 | Silent |
| w.t. | 680 | 40 | 22 | 61 | 44 | | | | | | | | | |
| 28C02 | 350 | 66 | 43 | 77 | 70 | N | L | F | F | S | | A | L | 0 |
| 29F09 | 215 | 87 | 69 | 38 | 8 | | L | F | F | S | S | A | L | 0 |
| mutBGL1 | 235 | | 65 | | 11 | N | L | F | F | S | S | A | L | 0 |

### Example 8

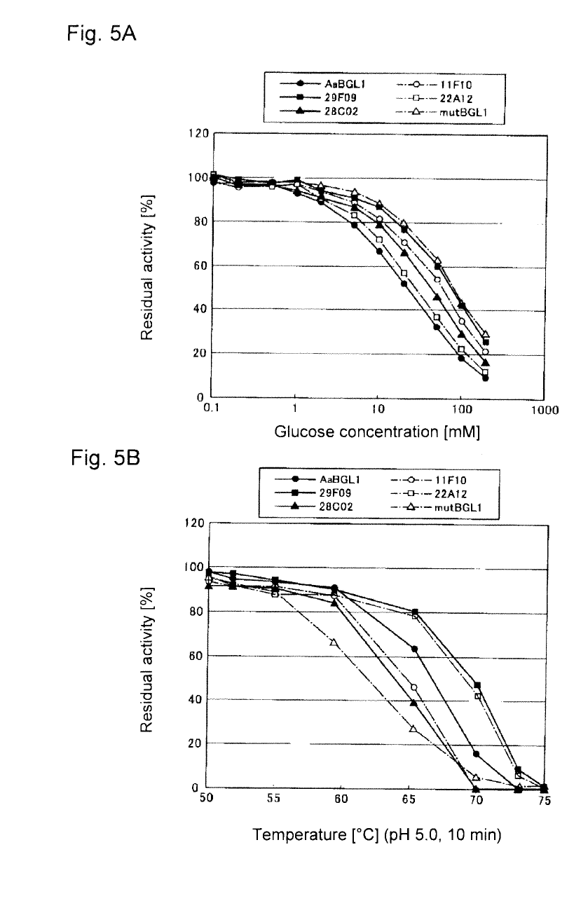

A mutant strain producing wild-type AaBGL1, 11F10 mutant strain, 22A12 mutant strain, 29F09 mutant strain, and 28C02 mutant strain were cultivated, and then the glucose inhibition and the thermostability at a warming temperature of from 50 to 75° C. of the culture supernatants of the respective mutant strains were measured. The results are shown in FIG. 5. In FIG. 5 (A), the glucosidase activity at a glucose concentration of 0 mM is set to 100%, and the residual glucosidase activities of the each culture supernatant at respective glucose concentrations are shown. In FIG. 5 (B), the glucosidase activity at a condition where preliminary heat treatment is not applied is set to 100%, and the residual glucosidase activities of the each culture supernatant at respective temperature conditions are shown.

### Example 9

It was suggested by the results of Examples 1 to 7 that Q142N (substitution of glutamine at position 142 to asparagine), Q156L (substitution of glutamine at position 156 to leucine), I120F (substitution of isoleucine at position 120 to phenylalanine), G298F (substitution of glycine at position 298 to phenylalanine), N458S (substitution of asparagine at position 458 to serine), G468S (substitution of glycine at position 468 to serine), V519A (substitution of valine at position 519 to alanine), and R629L (substitution of arginine at position 629 to leucine) are involved in the resistance to glucose inhibition or the thermostability of AaBGL1. Therefore, with regard to a mutant strain having P3AaBGL1 in which only one of these mutations was introduced, the resistance to glucose inhibition and the thermostability were measured.

### Preparation of Site-Specific Mutation Fragment

Firstly, mutant strains in which only Q156L, I120F, N458S, or R629L was introduced, were prepared.

Q156L specific amino acid substitution was carried out with primers. By using pSL6P3AaBGL1 as a template, a leu1⁺-Q156L fragment was amplified by PCR method with the primers of SEQ ID NO: 8 and SEQ ID NO: 26. On the other hand, a Q156L-top2 fragment was amplified by PCR method with the primers of SEQ ID NO: 27 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-Q156L-top2 fragment was amplified by PCR method with the primers of SEQ ID NO: 12 and SEQ ID NO: 13. Thereafter, a column purification was carried out by using SUPREC-PCR.

I120F specific amino acid substitution was carried out with primers. By using pSL6P3AaBGL1 as a template, a leu1⁺-I120F fragment was amplified by PCR method with the primers of SEQ ID NO: 8 and SEQ ID NO: 28. On the other hand, a I120F-top2 fragment was amplified by PCR method with the primers of SEQ ID NO: 29 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-I120F-top2 fragment was amplified by PCR method with the primers of SEQ ID NO: 12 and SEQ ID NO: 13. Thereafter, a column purification was carried out by using SUPREC-PCR.

N458S specific amino acid substitution was carried out with primers. By using pSL6P3AaBGL1 as a template, a leu1⁺-N458S fragment was amplified by PCR method with the primers of SEQ ID NO: 8 and SEQ ID NO: 30. On the other hand, a N458S-top2 fragment was amplified by PCR method with the primers of SEQ ID NO: 31 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-N458S-top2 fragment was amplified by PCR method with the primers of SEQ ID NO: 12 and SEQ ID NO: 13. Thereafter, a column purification was carried out by using SUPREC-PCR.

R629L specific amino acid substitution was carried out with primers. By using pSL6P3AaBGL1 as a template, a leu1⁺-R629L fragment was amplified by PCR method with the primers of SEQ ID NO: 8 and SEQ ID NO: 32. On the other hand, a R629L-top2 fragment was amplified by PCR method with the primers of SEQ ID NO: 33 and SEQ ID NO: 11.

To a reaction mixture comprised of these two types of fragments, restriction enzyme DpnI was added to digest the templates, and then the fragments were purified by using SUPREC-PCR for primer substitution. By using 50 ng of the purified two types of fragments, a leu1⁺-R629L-top2 fragment was amplified by PCR method with the primers of SEQ ID NO: 12 and SEQ ID NO: 13. Thereafter, a column purification was carried out by using SUPREC-PCR.

### Recovery of Mutant Strain Obtained by Site-Saturation Mutagenesis

Mutant strains were obtained in the same manner as in Example 1, except that each of the above-prepared fragments was used as a mutant gene-integrated fragment. Then, the nucleotide sequence of P3AaBGL1 gene integrated into the chromosomes of the each mutant strain obtained as above was confirmed.

### Evaluation of Glucosidase Activity, Resistance to Glucose Inhibition, and Thermostability

In the same manner as in Example 1, the glucose inhibition and the thermostability at a warming temperature of from 50 to 75° C. of the culture supernatants of the respective mutant strains were measured. The results are shown in Table 11.

TABLE 11

| | AaBGL1 | Q142N | Q156L | I260F | G298F | N458S | G468S | V519A | R629L | mutBGL1 |
|---|---|---|---|---|---|---|---|---|---|---|
| Activity | 594 | 533 | 507 | 457 | 278 | 586 | 244 | 426 | 526 | 191 |
| [mU/ml] | | | | | | | | | | |
| Glc [mM] | Residual glucosidase activity (%) | | | | | | | | | |
| 0 | 100 | 100 | 100 | 100 | 100 | 100 | 100 | 100 | 100 | 100 |
| 1 | 89.5 | 94.8 | 92.8 | 95.9 | 93.9 | 92.7 | 99.6 | 91.7 | 91.5 | 97.7 |
| 5 | 65.2 | 68.1 | 64.9 | 73.6 | 82.4 | 68.1 | 95.7 | 66.4 | 67.4 | 87.0 |
| 10 | 49.4 | 51.2 | 46.8 | 55.6 | 68.6 | 52.1 | 85.9 | 50.3 | 53.9 | 80.9 |
| 20 | 33.4 | 35.7 | 31.5 | 39.0 | 53.8 | 35.9 | 76.1 | 35.4 | 37.6 | 72.6 |
| 50 | 17.6 | 17.8 | 15.5 | 20.2 | 32.4 | 17.7 | 55.8 | 17.7 | 19.6 | 57.7 |
| 100 | 10.5 | 10.1 | 8.8 | 11.4 | 19.9 | 10.4 | 36.6 | 9.6 | 11.1 | 39.5 |
| 200 | 5.1 | 5.8 | 5.4 | 6.3 | 10.9 | 6.0 | 21.4 | 5.7 | 6.9 | 27.0 |
| Temp [° C.] | Residual glucosidase activity (%) | | | | | | | | | |
| Untreated | 100 | 100 | 100 | 100 | 100 | 100 | 100 | 100 | 100 | 100 |
| 50 | 96.4 | 96.1 | 93.9 | 97.0 | 89.5 | 98.6 | 93.1 | 95.6 | 98.7 | 93.4 |
| 55 | 91.5 | 91.1 | 89.7 | 91.2 | 83.4 | 95.7 | 88.6 | 90.3 | 93.7 | 91.0 |
| 60 | 90.4 | 89.0 | 83.9 | 87.7 | 75.7 | 91.6 | 63.0 | 88.1 | 90.7 | 82.5 |
| 65 | 60.0 | 58.7 | 46.0 | 65.2 | 38.2 | 64.2 | 11.8 | 72.8 | 71.8 | 37.4 |
| 70 | 16.0 | 15.4 | 6.3 | 22.6 | 4.3 | 19.4 | 1.0 | 26.5 | 26.5 | 0.5 |
| 73 | 1.1 | 1.2 | 0.9 | 1.8 | 1.5 | 0.8 | 1.4 | 2.4 | 3.5 | 1.4 |
| 75 | 1.5 | 1.2 | 1.4 | 1.8 | 1.8 | 1.6 | 1.7 | 1.7 | 1.5 | 3.3 |

As a result, I260F was found to increase both the resistance to glucose inhibition and the thermostability of P3AaBGL1. Further, G298F and G468S were found to increase the resistance to glucose inhibition and decrease the thermostability of P3AaBGL1. Further, it was found that the thermostability can be improved without decreasing the resistance to glucose inhibition of P3AaBGL1 by N458S, V519A or R629L. Further, Q156L was found to decrease both the resistance to glucose inhibition and the thermostability of P3AaBGL1.

Among these mutation sites of P3AaBGL1, glycine at position 468 and valine at position 519 are highly preserved in various β-glucosidases. Further, the amino acids correspond to isoleucine at position 260, glycine at position 298, asparagine at position 458 or arginine at position 629 share similar size of side chain and polarity among various β-glucosidases, and the upstream and downstream regions of the amino acids are highly preserved among various β-glucosidases. Accordingly, it is highly probable that all of the above-mentioned amino acids are important for the activity of β-glucosidase, and therefore the activities of various β-glucosidases seemed to be affected in the same manner as P3AaBGL1 described in Examples, by substituting these amino acids in the same manner as for P3AaBGL1.

### Example 10

### Purification of Modified-Type P3AaBGL1

11F10 strain, 22A12 strain, 28C02 strain, 29F09 strain and mutBGL1 strain selected from the mutant strains, and ASP3326 strain as the original strain were cultivated in a Sakaguchi flask containing 100 ml of YPD medium at 30° C. for two days. To each of the culture supernatants obtained by cultivating them, ammonium acetate was added until 80% saturation was reached, thereby to carry out ammonium sulfate precipitation at 4° C. for 2 hours. Thereafter, centrifugation was carried out to collect pellets, and the pellets were dissolved in 10 ml of 10 mM citrate phosphate buffer solution (pH 7.0). Then, desalting and concentration were carried out by using an ultrafiltration membrane having MWCO of 100,000, thereby to recover 1 ml of a crude liquid. Thus recovered crude liquid was purified by using Hitrap QXL column to collect a fraction of a peak of pNPG activity, thereby to obtain a purified sample. Elution was carried out by using a 10 mM citrate phosphate buffer solution (pH 7.0) and a NaCl concentration gradient (0 to 1M).

### Characterization of the Purified Modified-Type P3AaBGL1

In the same manner as in Example 1, the residual glucosidase activities after glucose inhibition at a glucose concentration of from 0.1 to 200 mM were measured. Further, the residual glucosidase activities after heat treatment at a temperature of from 50 to 75° C. for 10 minutes were measured. As a result, a glucose inhibition resistance improved-type P3AaBGL1 of 11F10 strain was found to maintain 53% of the activity in the presence of 50 mM glucose. A thermostability improved-type P3AaBGL1 of 22A12 strain was found to maintain around 80% of the residual activity at 65° C. Further, a glucose inhibition resistance and thermostability improved-type P3AaBGL1 of mutBGL1 strain was found to maintain 60% of the activity in the presence of 50 mM glucose and around 45% of the residual activity at 65° C.

### INDUSTRIAL APPLICABILITY

The β-glucosidase produced from the transformant of the present invention can be used for the enzymatic saccharification of cellulose. Further, when the transformant of the present invention is a transformant which can produce a β-glucosidase having a secretion signal, by cultivating the transformant in a culture broth containing cellulose, it is possible to utilize cellulose contained in the culture broth by the enzymatic saccharification, since the β-glucosidase is secreted into the culture broth.

This application is a continuation of PCT Application No. PCT/JP2012/057055, filed on Mar. 19, 2012, which is based upon and claims the benefit of priority from Japanese Patent Application No. 2011-066539 filed on Mar. 24, 2011. The contents of those applications are incorporated herein by reference in its entirety.

What is claimed is:

1. A transformant of a yeast of the genus Schizosaccharomyces characterized by having a structural gene sequence comprising a region encoding the following modified-type β-glucosidase, and a promoter sequence and a terminator sequence for expressing the structural gene in a chromosome, or alternatively by having the sequences as an extrachromosomal gene.
Modified-type β-glucosidase: A β-glucosidase derived from a filamentous fungus having at least one mutation selected from the group consisting of the following mutations 1 to 6, and has higher thermostability or higher resistance to glucose inhibition than a β-glucosidase which does not have the mutations.
Mutation 1; a mutation in which an amino acid corresponds to isoleucine at position 244 of AaBGL1 (BGL1 of Aspergillus aculeatus) is substituted by phenylalanine.
Mutation 2; a mutation in which an amino acid corresponds to glycine at position 282 of AaBGL1 is substituted by phenylalanine.
Mutation 3; a mutation in which an amino acid corresponds to asparagine at position 442 of AaBGL1 is substituted by serine.
Mutation 4; a mutation in which an amino acid corresponds to glycine at position 452 of AaBGL1 is substituted by serine.
Mutation 5; a mutation in which an amino acid corresponds to valine at position 503 of AaBGL1 is substituted by alanine.
Mutation 6; a mutation in which an amino acid corresponds to arginine at position 613 of AaBGL1 is substituted by leucine.

2. The transformant of a yeast of the genus Schizosaccharomyces according to claim 1, wherein the β-glucosidase derived from a filamentous fungus is BGL1.

3. The transformant of a yeast of the genus Schizosaccharomyces according to claim 1, wherein the filamentous fungus is a microorganism of the genus Aspergillus.

4. The transformant of a yeast of the genus Schizosaccharomyces according to claim 1, wherein the β-glucosidase derived from a filamentous fungus is comprised of an amino acid sequence having at least 70% homology with an amino acid sequence of SEQ ID NO: 1, and is a protein has a catalytic activity to hydrolyze a β-D-glucopyranoside bond.

5. The transformant of a yeast of the genus Schizosaccharomyces according to claim 1, wherein the structural gene sequence further comprises a region encoding a signal or a region encoding a tag at the 5′ end side or the 3′ end side of the region encoding the modified-type β-glucosidase.

6. The transformant of a yeast of the genus Schizosaccharomyces according to claim 1, wherein the structural gene sequence further comprises a region encoding a secretion signal at the 5′ end side of the region encoding the modified-type β-glucosidase.

7. A transformation method for a yeast of the genus Schizosaccharomyces, characterized in that the yeast of the genus Schizosaccharomyces is transformed by using a vector containing a structural gene sequence comprising a region encoding the following modified-type β-glucosidase, and a promoter sequence and a terminator sequence for expressing the structural gene.
Modified-type β-glucosidase: A β-glucosidase derived from a filamentous fungus having at least one mutation selected from the group consisting of the following mutations 1 to 6, and has higher thermostability or higher resistance to glucose inhibition than a β-glucosidase which does not have the mutations.
Mutation 1; a mutation in which an amino acid corresponds to isoleucine at position 244 of AaBGL1 (BGL1 of Aspergillus aculeatus) is substituted by phenylalanine.
Mutation 2; a mutation in which an amino acid corresponds to glycine at position 282 of AaBGL1 is substituted by phenylalanine.
Mutation 3; a mutation in which an amino acid corresponds to asparagine at position 442 of AaBGL1 is substituted by serine.
Mutation 4; a mutation in which an amino acid corresponds to glycine at position 452 of AaBGL1 is substituted by serine.
Mutation 5; a mutation in which an amino acid corresponds to valine at position 503 of AaBGL1 is substituted by alanine.
Mutation 6; a mutation in which an amino acid corresponds to arginine at position 613 of AaBGL1 is substituted by leucine.

8. The transformation method for a yeast of the genus Schizosaccharomyces according to claim 7, wherein the structural gene sequence further comprises a region encoding a signal or a region encoding a tag at the 5′ end side or the 3′ end side of the region encoding the modified-type β-glucosidase.

9. The transformation method for a yeast of the genus Schizosaccharomyces according to claim 7, wherein the structural gene sequence further comprises a region encoding a secretion signal at the 5′ end side of the region encoding the modified-type β-glucosidase.

10. The transformation method for a yeast of the genus Schizosaccharomyces according to claim 7, wherein the vector is integrated into at least one position of a chromosome of the yeast of the genus Schizosaccharomyces.

11. The transformation method for a yeast of the genus Schizosaccharomyces according to claim 10, wherein the vector is integrated into a transposon gene Tf2 site of the chromosome.

12. A method for producing a β-glucosidase, characterized in that the modified-type β-glucosidase is recovered from cells obtained by cultivating the transformant as defined in claim 1.

13. A method for producing a β-glucosidase, characterized in that the modified-type β-glucosidase is recovered from a culture supernatant obtained by cultivating the transformant as defined in claim 6.

14. A cellulose degradation method, characterized in that the modified-type β-glucosidase obtained by the production method as defined in claim 12 is used.

15. A cellulose degradation method, characterized in that the transformant as defined in claim 6 is cultivated in the presence of cellulose.
